(19) United States
(12) Patent Application Publication
(10) Pub. No.: US 2010/0248260 A1
(43) Pub. Date: Sep. 30, 2010

(54) GLYCOSAMINOGLYCAN-COATED PARTICLES AND USES THEREOF

(75) Inventors: Zhihui BAN, Acton, MA (US); Carlos Bosques, Cambridge, MA (US); Ram Sasisekharan, Cambridge, MA (US)

Correspondence Address: CHOATE, HALL & STEWART LLP, TWO INTERNATIONAL PLACE, BOSTON, MA, 02110, US

(73) Assignee: MASSACHUSETTS INSTITUTE OF TECHNOLOGY, Cambridge, MA (US)

(21) Appl. No.: 12/746,033

(22) PCT Filed: Dec. 5, 2008
(86) PCT No.: PCT/US08/13387

§ 371 Date: Jun. 18, 2010

Related U.S. Application Data
(60) Provisional application No. 60/992,630, filed on Dec. 5, 2007.

(51) Int. Cl. G01N 33/53; B32B 1/00

(52) U.S. Cl. 435/7.1; 428/403

(57) ABSTRACT

The present invention is directed to metallic particles coated with glycosaminoglycans and methods for preparing them. Methods of using such glycosaminoglycan-coated metallic particles in biomedical and other applications are also disclosed. In certain embodiments, methods for assaying glycosaminoglycan-degrading activity in biological fluids, test samples, and/or therapeutic formulations using the glycosaminoglycan-coated particles are provided. Such methods may be used, for example, in diagnostic tests for diseases such as cancer, inflammatory diseases, or autoimmune diseases and to test activity of enzymes being developed as therapeutics.

### RELATED APPLICATION

This application claims priority under 35 U.S.C. §119(e) to U.S. provisional patent application, U.S. Ser. No. 60/992,630, filed Dec. 5, 2007, which is incorporated herein by reference.

### GOVERNMENT SUPPORT

This invention was made with U.S. government support under the National Institute of General Medical Sciences Grant No. GM57073 and the National Institutes of Health/National Institute of Environmental Health Sciences Grant No. 5-T32-ES0720 awarded by the National Institutes of Health. The U.S. government has certain rights in the invention.

### BACKGROUND OF THE INVENTION

Glycosaminoglycans are long unbranched chains of polysaccharides consisting of repeating disaccharide units. Glycosaminoglycans are a highly abundant class of molecules that play a variety of important roles in cellular biology. For example, they are a major component of the extracellular matrix and are involved in cell adhesion, migration, and signaling. (See Anat Rec. 2002; 268(3):317-26: “Evolution of Glycosaminoglycans and Their Glycosyltransferase: Implications for the Extracellular Matrices of Animals and the Capsules of Pathogenic Bacteria.”) Glycosaminoglycans are usually covalently linked to proteins, thereby forming proteoglycans. Proteoglycans are involved in biological processes such as cell signaling and movement of molecules through the extracellular matrix.

Glycosaminoglycan-degrading enzymes have recently been implicated in human disease. Heparanase activity has been found to be consistently elevated in multiple myeloma, suggesting that heparanase activity could be a reliable diagnostic indicator of the disease. See Cancer Res. 2003 Dec. 15; 63(24):8749-56.) Similarly, elevated hyaluronidase activity is associated with prostate cancer and bladder cancer. See, for example, Cancer Res. 2005 Sep. 1; 65(17): 7782-7789) Given the role of glycosaminoglycan-degrading enzymes in disease processes, sensitive assays for such activity in a biological sample would be useful in the diagnosis and following the treatment of such diseases.

### SUMMARY OF THE INVENTION

The development of potential diagnostic tools using glycosaminoglycan-degrading activity as an indicator has been hampered by the difficulty of measuring the activity of glycosaminoglycan-degrading enzymes in biological environments such as in the presence of bodily fluids and/or cellular components. Current methods for measuring glycosaminoglycan-degrading activity are difficult to employ directly in biological environments.

Microscopic particles such as nanoparticles have been developed for use in many biological and clinical applications. In particular, metallic particles such as those made from gold, cadmium sulfide, and lead sulfide have advantageous optical and electrical properties that lend to their use in biosensing applications. Complex carbohydrates, including glycosaminoglycans, have a propensity to degrade under harsh conditions and have limited reactive groups for derivatization and attachment to such particles. Thus, it is difficult to manipulate glycosaminoglycans and other complex carbohydrates such that they can be combined with useful reagents such as, metallic particles. Also, synthesizing metallic particles that are water soluble is often difficult. Methods of synthesizing water-soluble metallic particles coated with glycosaminoglycans would be advantageous. Such glycosaminoglycan-coated particles would be particularly useful in methods for detecting and/or monitoring glycosaminoglycan-degrading activity.

In some aspects, the present invention provides metallic particles coated with glycosaminoglycans useful in the diagnosis of diseases such as cancer by monitoring glycosaminoglycan-degrading activity. The glycosaminoglycan-coated particles are also useful for monitoring enzyme activity levels for diagnostic purposes or for following treatment. Metallic particles provided in certain embodiments have on average a largest dimension (e.g., the diameter for a spherical particle) of less than 1 μm and are typically referred to as nanoparticles. Any metal may be used to prepare the particles as long as the resulting particles have the required optical and/or electrical properties for detecting glycosaminoglycan-degrading activity. In certain embodiments, the metallic particles are made of gold, cadmium sulfide, or lead sulfide. Any glycosaminoglycan or complex carbohydrate, the degradation of which is to be measured, may be used on the surface of the inventive particles. In certain embodiments, the glycosaminoglycan coating the metallic particles is chondroitin, chondroitin sulfate, dermatan, dermatan sulfate, keratan, keratan sulfate, heparin, heparan sulfate, or hyaluronan.

In some aspects, the present invention provides methods of synthesizing metallic particles coated with glycosaminoglycans. In certain embodiments of the invention, methods comprise mixing gold precursors (e.g., gold salts) with glycosaminoglycans in water, optionally heating the solution, and then adding a reducing agent such as citric acid or a citrate salt to the gold-glycosaminoglycan solution with stirring, thereby generating soluble gold particles coated with glycosaminoglycans. In certain embodiments of the invention, the particles are soluble glycosaminoglycan-coated cadmium sulfide particles. In some such embodiments, methods comprise mixing cadmium precursors (e.g., Cd(NO₃)₂) with glycosaminoglycans in water and then adding a sulfide solution (e.g., NaS₂) to the mixture. In certain embodiments of the invention, the particles are soluble lead sulfide particles. In such embodiments, methods comprise mixing lead precursors (e.g., Pb(NO₃)₂) with glycosaminoglycans and adding a sulfide solution to the mixture to produce soluble lead sulfide particles coated with the glycosaminoglycan. In some embodiments, the reducing capacity of the glycosaminoglycan is not used, i.e., to interact with the metal precursor.

In another aspect, the present invention provides methods of using such particles in assays for monitoring the activity of glycosaminoglycan-degrading enzymes. (See FIG. 1 for a general schematic.) In some embodiments, the glycosaminogycan-degrading enzyme that is monitored is being produced, for example, for commercial and/or therapeutic purposes. In some embodiments, the glycosaminoglycan-degrading enzyme that is monitored is relevant to a human disease. For example, the glycosaminoglycan coating the particle may be a glycosaminoglycan that is degraded by an enzyme secreted by a cancer cell or an inflammatory cell. The activity of the glycosaminoglycan-degrading enzyme in a sample (e.g., a biological sample like blood, serum, ascites, etc.) can be monitored using any of the disclosed methods.

For example, some embodiments of the invention provide metallic particles coated with heparan sulfate, which is degraded by heparanase, an enzyme associated with multiple myeloma. Such particles can be used to determine disease progression in multiple myeloma patients. A biological sample from a patient is contacted with heparan sulfate-coated metallic particles under suitable conditions to allow for heparan sulfate on the surface of the particles to be degraded by heparanase activity present in the biological sample.

Degradation of heparan sulfate on the surface of the particles allows aggregation of metallic particles The aggregation of the particles can be detected using spectrocopic techniques, for example by determining a shift in the visible spectrum of the particles. Biological samples [symbol] urine, saliva, synovial fluid, cerebrospinal fluid, tear fluid, vitreous humor, venom, cell supernatant, cell lysate, and cell culture medium.

In some aspects, the invention provides kits that may be useful in the diagnosis and/or monitoring of diseases involving glycosaminoglycan-degrading activity. In some embodiments of the invention, the kit comprises metallic particles coated with glycosaminoglycans, at least one control sample having glycosaminoglycan-degrading activity (i.e., a positive control), and instructions for use. In some embodiments of the invention, the kit also comprises at least one control sample that does not have glycosaminoglycan-degrading activity (i.e., a negative control).

These and other features of the present invention will become apparent to those of ordinary skill in the art having read the following detailed description of the certain embodiments of the invention.

This application refers to various patents and publications. The contents of all of these are incorporated by reference. In case of a conflict between the instant specification and one or more of the incorporated references, the specification shall control.

### BRIEF DESCRIPTION OF THE DRAWINGS

FIG. 1 depicts a schematic representation showing gold nanoparticles with glycosaminoglycans (at left) obtained by template-assisted synthesis methods provided herein. Aggregation of gold nanoparticles (GNPs) (at right) occurs in the presence of glycosaminoglycan-degrading enzyme activity.

FIG. 2 depicts glycosaminoglycan-assisted synthesis of soluble gold nanoparticles. Depicted are products of reactions between precursors when (A) HAuCl₄ (a gold precursor) is mixed with 1% w/v Na₃C₆H_{S}O₇ (a gold precursor) in the absence of heparan sulfate (B) HAuCl₄ is mixed with Na₃C₆H_{S}O₇ followed by the addition of heparan sulfate, and (C) HAuClₐ is first mixed with heparan sulfate followed by the addition of trisodium citrate.

FIG. 3 depicts photophysical alterations to heparan sulfate-coated gold nanoparticles upon treatment with Heparinase III enzyme. (A) Visible inspection and (B) absorbance spectra of the solution before (purple) and after (blue) treatment with the enzyme. (C) Change in absorbance maxima as a function of enzyme treatment time. (D) Change in absorbance intensity at the wavelength maximum for heparan sulfate gold nanoparticles as a function of time after addition of 1 μg heparinase.

FIG. 4 shows plots depicting changes in (A) wavelength and (B, C) absorbance intensity for heparan sulfate gold nanoparticles as a function of heparinase activity. In (B), change in the absorbance intensity at the wavelength maximum for each heparinase amount is plotted. In (C), FIG. 4C shows changes in the absorbance intensity at 630 nm plotted.

FIGS. 5A and 5B depict transmission electron micrographs of heparan sulfate-coated gold nanoparticles (A) before and (B) after treatment with Heparinase III enzyme. Scale bars represent 20 nm.

FIG. 5C depicts a schematic representation of the aggregation of the gold nanoparticles (GNPs) upon enzyme treatment.

FIG. 6 depicts photophysical alterations to heparan sulfate-coated gold nanoparticles before (purple) and after (blue) exposure to Heparinase III in (A) serum and (B) plasma.

FIG. 7 depicts absorbance spectra for heparan sulfate-GNP in a variety of samples. For each sample, 1 mL of serum (purple), 1 mL of plasma (red), 1 mL of 10% w/v BSA (blue), or 1 mL of PBS buffer pH 7.4 (green) were added to a solution containing 1.5 mL of 51.2 μg/mL heparan sulfate-GNP in water and 0.4 mL of PBS buffer pH 7.4. Spectra for heparan sulfate-GNP in buffer alone are shown in blue.

FIG. 8 depicts glycosaminoglycan- and oligosaccharide-assisted synthesis of soluble cadmium sulfide (CdS) nanoparticles. Depicted are products of reactions between precursors (A) without any kind of polysaccharide (B) with the assistance of heparan sulfate I and (C) with the assistance of chitosan oligosaccharide lactate.

FIG. 9 shows the morphology and dimensions of glycosaminoglycan-coated CdS nanoparticles. Shown are high resolution transmission electron micrograph (TEM) images of CdS nanoparticles prepared in water with different polysaccharides. FIG. 9A depicts nanoparticles prepared with oligosaccharide lactate at 400× magnification. The scale bar represents 5 nm and the average size of the nanoparticles is 4 nm±0.3 nm. The inset corresponds to the selected area electron diffraction (SAED) pattern of the CdS nanoparticles. FIG. 9B depicts an enlarged image for the black square in FIG. 9A. FIG. 9C depicts nanoparticles prepared with heparan sulfate I at 400× magnification. The scale bar represents 5 nm and the average size of the nanoparticles is 4 nm±0.3 nm. The inset corresponds to the SAED pattern of the CdS nanoparticles. FIG. 9D depicts an enlarged image for the black square in FIG. 9C. In FIGS. 9B and 9D, the crystalline of [symbol] FIG. 9E depicts a typical energy dispersive analysis of X-ray (EDAX) spectrum of the synthesized CdS nanoparticles.

FIG. 10 demonstrates the morphological change of CdS nanoparticles at higher temperature and depicts TEM images of CdS nanoparticles that were annealed further for 60 minutes at 60° C., (A) at 400× magnification and (B) at higher resolution. In FIG. 10B, note that the crystallinity of the nanoparticles is clear in each aggregated particle.

FIG. 11 shows optical properties of CdS/heparan sulfate synthesis products. Depicted are fluorescence spectra of CdS nanoparticles synthesized with heparan sulfate I in water (A) as synthesized and (B) after being annealed at 60° C. for 60 minutes. The wavelength of excitation was 400 nm. In (A), the emission peak was at 490 nm (2.64 eV) and in (B), the emission peak was at 520 nm (2.5 eV).

FIG. 12 illustrates two processes used for the synthesis of lead sulfide (PbS) nanoparticles. In process #1, Pb²⁺ and S²⁻ are reacted together first, then polysaccharides are added to the solution. In process #2, a chosen ligand-polysaccharide is seeded with Pb²⁺ first, then an S²⁻ source is added to the solution.

FIG. 13 demonstrates the solubility in water of reaction products synthesized using different processes and polysaccharides. Depicted are photographs of reaction tubes taken immediately after syntheses. (1) denotes reactions carried out by process #1. (That is, the polysaccharide was added after the mixture of precursors Pb(NO₃)₂ and Na₂S.) (2) denotes reactions carried out by process #2. (That is, the polysaccharide was mixed with Pb(NO₃)₂ first, then Na₂S was added.) From left to right, the reactions were carried out with galactose, dextran sulfate B, chondroitin 6-sulfate, chondroitin acid, pectin, and hyaluronan. The right-most tube in the figure contains a reaction without any polysaccharide.

FIGS. 14A-G show TEM images of aggregated PbS nanoparticles synthesized with various capping agents. Shown are PbS nanoparticles synthesized (A) without capping, (B) capped with galactose, (C) capped with pectin, (D) capped with chondroitin 6-sulfate, (E) capped with dextran, (F) capped with hyaluronan, and (G) capped with heparin.

FIG. 14H shows a high resolution TEM image of a single PbS nanoparticle coated with hyaluronan, as in FIG. 14F.

FIG. 14I demonstrates that the synthesized PbS nanoparticles are indeed composed of Pb²⁺ and S²⁻. Shown is a typical EDAX spectrum of synthesized PbS nanoparticles. The Cu represented by the second, fourth, and fifth peaks from the left is from the grid.

FIG. 15 shows optical properties of crystals of PbS nanoparticles coated with hyaluronic acid, chondroitin 6-sulfate, and dextran. Shown on the left side is the UV-Vis spectrum. Shown on the right side is photoluminescence spectra (black: hyaluronic acid; red: chondroitin-6 sulfate; green: dextran). All of the curves have been normalized to the maximum.

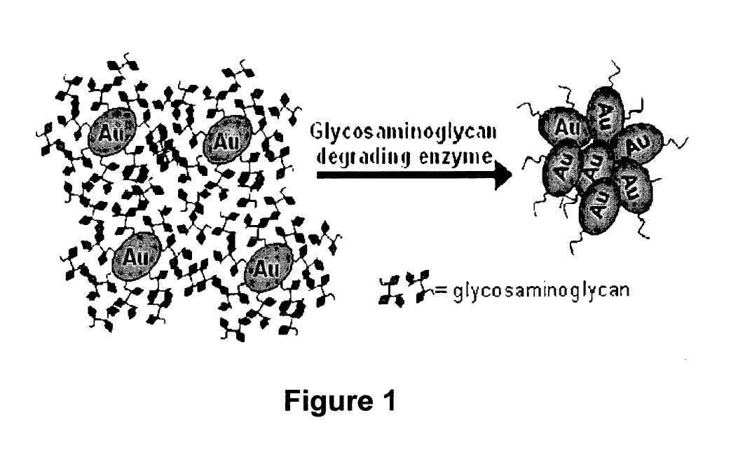

### DEFINITIONS

The phrase “ascites fluid,” is used herein to refer to fluid that accumulates in the peritoneal cavity. The presence of ascites fluid in a subject often leads to abdominal distension and may be indicative of a medical problem or condition. Ascites fluid occurs commonly in cirrhosis, though it is also present in other diseases such as cancer, heart failure, kidney failure, and pancreatitis.

The term “aggregate,” when used as a noun, is used herein to refer to a mass or body of units or parts somewhat loosely associated with one another. When used as a verb, the term “aggregate” is used herein to mean to collect or gather into a mass or whole.

The terms “approximately” and “about” in reference to a number include numbers that fall within a range of 20%, 10%, 5%, 2%, or 1% in either direction (greater than or less than) the number unless otherwise stated or otherwise evident from the context (except where such number would exceed 100% of a possible value.)

The term “assay” is used herein to refer to a procedure where a property or a concentration of a molecule such as an enzyme is measured. As used herein, to “assay” something means to test for the presence of, and/or measure a property or a concentration of a molecule. In certain embodiments, “assay” as used herein may refer to measuring glycosaminoglycan-degrading activity in a sample.

The phrase “autoimmune disease” refers to any disease resulting from an aberrant immune response of an organism against its own cells and/or tissues. “Autoimmune diseases” often stem from a failure of the organism to recognize its own substituents (such as cells, tissues, proteins, and molecules) as “self.” Exemplary autoimmune diseases include rheumatoid arthritis, diabetes mellitus type 1, systemic lupus erythrematosis, Sjögren's disease, multiple sclerosis, Hashimoto's thyroiditis, Grave's disease, Coeliac disease, and idiopathic thrombocytopenic purpura.

The term “cancer” refers to or describes the physiological condition in mammals that is typically characterized by unregulated cell growth. Examples of cancers include, but [symbol] particularly, examples of such cancers include multiple myeloma, prostate cancer, lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma.

The term “cancer cell” refers to a cell in a subject (e.g., a human being) in vivo which undergoes undesired and unregulated cell growth or abnormal persistence or abnormal invasion of tissues. In vitro, this term also refers to a cell line that is a permanently immortalized established cell culture that will proliferate indefinitely and in an unregulated manner given appropriate fresh medium and space.

The term “carbohydrate” is used herein to refer to a sugar or polymer of sugars. The terms “saccharide”, “polysaccharide”, “carbohydrate”, and “oligosaccharide”, may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

The phrase “cell culture,” is used herein to refer to the growing of cells, typically [symbol] especially animal cells, or can be microorganisms such as bacteria. The term “tissue culture” is often used interchangeably with the term “cell culture” when the cells are derived from multicellular eukaryotic animals.

The phrase “cell culture medium,” (sometimes referred to as “medium”) is used herein to refer to the substance in which cells are grown. There are a variety of types of cell culture medium that come in liquid and other forms, such as soft agars. Cell culture medium typically contains nutrients that facilitate cell growth. Liquid media for growing cells that are not microorganisms, such as mammalian cells, typically also contain growth factors such as that can be found in serum.

The phrase “cell lysate” is used herein to refer to the solution produced when cells are destroyed by disrupting the cellular membrane, containing cellular proteins, nucleic acids, organelles, and/or lipids. As used herein, the phrase “cell lysate” refers to both the crude solution produced after cellular rupture and a form of that solution purified or separated by means such as centrifugation.

The term “chondroitin sulfate” is used herein to refer to a glycosaminoglycan comprised of sulfated GlcA-GalNAc disaccharide units linked together by via alternating β-1,4 and β-1,3 glycosidic bonds and usually found covalently linked to a protein. The extent and location of sulfation along chondroitin sulfate chains vary. Common positions of sulfation include the C4 and C6 positions of GalNAc residues and the C2 position of GlcA residues. Chondroitin sulfate is the most abundant glycosaminoglycan and is found in cartilage, bone, and heart valves. The term “chondroitin” is used herein to refer to a glycosaminoglycan similar in structure to chondroitin sulfate, but lacking in sulfation in most or all residues. Typical structures for the repeating disaccharide unit of which chondroitin sulfate is comprised are shown below for two common variants of chondroitin sulfate, chondroitin 4-sulfate and chondroitin 6-sulfate:

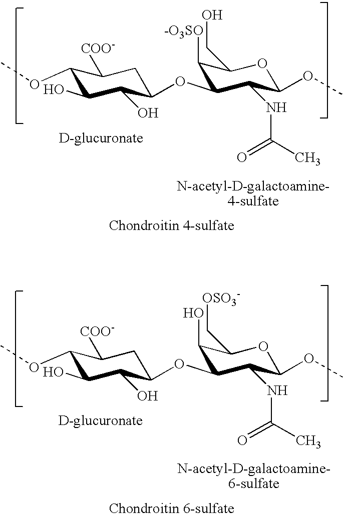

The term “coat” is used herein as a verb to mean cover the surface of something (e.g., a particle). As used herein, molecules that are said to coat the surface of a particle do not necessarily cover the entire surface of the particle, nor are they necessarily restricted to the surface of the particle. Rather, the term “coat” is used to mean that such molecules are present on at least a portion of the surface of the particles and are exposed to the environment external to the particles.

The abbreviation “Da” is used herein to refer to a dalton, a unit of mass also known as a unified atomic mass unit, defined as one twelfth of the mass of an unbound atom of the carbon-12 nuclide, at rest and its ground state. The abbreviation “kDa” is used herein to refer to a kilodalton, equivalent to 1000 daltons.

The term “degrade,” is used herein to mean to reduce the complexity of (a chemical compound) by splitting off one or more groups or larger components. In certain embodiments of the invention, “to degrade” a glycosaminoglycan means to break up the chain of disaccharide units, thereby depolymerizing it into monosaccharides, disaccharides, oligosaccharides, and low molecular weight polysaccharides.

The term “dermatan sulfate” is used herein to refer to a glycosaminoglycan also known as “chondroitin sulfate B” and comprised of sulfated IdoA-GalNAc disaccharide units linked together via alternating β-1,4 and β-1,3 glycosidic bonds and commonly found covalently linked to a protein. Dermatan sulfate is found mostly in skin, but also in blood vessels, heart valves, tendons, and lungs. Dermatan sulfate proteoglycans may have roles in coagulation and wound repair, and have also been implicated in cardiovascular disease, carcinogenesis, infection, and fibrosis. The term “dermatan” is used herein to refer to a glycosaminoglycan similar in structure to dermatan sulfate, but lacking in sulfation in most or all residues. A typical structure for the repeating disaccharide unit of which dermatan sulfate is comprised is shown below:

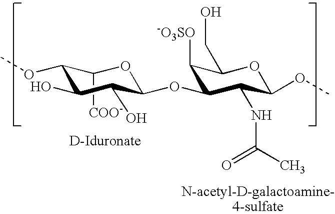

The term “disaccharide” is used herein to refer to a carbohydrate composed of two monosaccharides.

The term “epimer” is used herein to refer to a stereoisomer of another compound that has a different configuration at only one of several stereogenic centers.

The term “epimerisation” is used herein to refer to the interconversion of epimers by reversal of the configuration at one of the stereogenic centers.

The abbreviation “Gal” is used herein to refer to galactose.

The abbreviation “GalNAc” is used herein to refer to N-acetylgalactosamine.

The abbreviation “GalNAc(6S)” is used herein to refer to N-acetylgalactosamine sulfated at the C6 position.

The abbreviation “GlcA” is used herein to refer to glucoronic acid.

The abbreviation “GlcN” is used herein to refer to glucosamine.

The abbreviation “GlcNAc” is used herein to refer N-acetylglucosamine.

The abbreviation “GlcNS” is used herein to refer to N-sulfated glucosamine.

The abbreviation “GlcNS(6S)” is used herein to refer to N-sulfated glucosamine that is also sulfated at the 06 position.

The term “glycan” is used interchangeably with “saccharide” and “carbohydrate” and is used herein to refer to any sugar or assembly of sugars, in free form or attached to another molecule.

The term “glycosaminoglycan” (abbreviated as “GAG”) is used herein to refer to a class of linear unbranched polysaccharides comprising a repeating disaccharide unit, which typically comprise hexosamine and a hexose or a hexuronic acid. In some embodiments, the repeating disaccharide unit comprises a glucosamine or galactosamine followed by an iduronic or glucoronic acid. Glycosaminoglycans are typically highly negatively charged and have special structural features that contribute to their various functions. For example, in an extended conformation, glycosaminoglycans contribute to the viscosity of the fluid of which they are a part. Their rigidity provides structural integrity that is central to their role in cell migration. Glycosaminoglycans are the most abundant heteropolysaccharides in the body, forming a major component of the extracellular matrix as well as a major part of glycoproteins commonly found on the cell surface. Glycosaminoglycans are often covalently attached to proteins, forming together with the protein a proteoglycan.

Examples of glycosaminoglycans include, but are not limited to, chondroitin sulfate, dermatan sulfate, keratan sulfate, heparin, heparan sulfate, and hyaluronan. Those of ordinary skill in the art will appreciate that there is structural variability in the chemical structures within one type of glycosaminoglycan (such as heparin). Though glycosaminoglycans are recognizable by a general chemical structural theme, a glycosaminoglycan of a given type exists in multiple forms and varies in the composition of disaccharide units that comprise it. They also vary in the extent and pattern of sulfation along the molecule. Nevertheless, the term “glycosaminoglycan” will be understood by those of ordinary skill in the art to mean a particular class of polysaccharides as described above.

The phrase “glycosaminoglycan-degrading activity” is used herein to refer to any activity that breaks up the chain of disaccharide units that form glycosaminoglycans, thereby depolymerizing glycosaminoglycans into monosaccharides, disaccharides, oligosaccharides, and/or low molecular weight polysaccharides.

The phrase “glycoside hydrolase” is used herein to refer to a group of enzymes that hydrolyse the glycosidic bond between two or more carbohydrates or between a carbohydrate and a non-carbohydrate moiety.

The term “heparan sulfate” (also known as heparatan sulfate, heparin monosulfate, heparatin, heparitin monosulfate, and heparatin sulfate) is used herein to refer to a glycosaminoglycan comprised of sulfated GlcA-GlcNAc disaccharide units linked together via α-1,4 glycosidic bonds and typically found covalently linked to a protein. GlcA-GlcNAc disaccharide units commonly comprise about 50% of the total number of disaccharide units in heparan sulfate. Other disaccharide units found in heparan sulfate include GlcA-GlcNS and IdoA-GlcNS. Sulfation at IdoA residues typically occurs at C2 position, and sulfation at GlcNS residues typically occurs at the C6 position. One or both [symbol] 3-O-sulfated glucosamine (GlcNS(3S,6S)) or a free amine group (GlcNH₃⁺) are also found in heparan sulfate. As part of a proteoglycan, heparan sulfate is known to be involved in developmental processes, angiogenesis, blood coagulation, and tumor metastasis. A typical structure for a portion of a heparan sulfate chain is shown below:

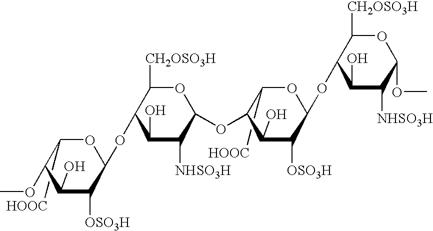

The term “heparin” is used herein to refer to a glycosaminoglycan comprised mostly of sulfated IdoA-GlcNS disaccharide units linked together via α-1,4 glycosidic bonds. Sulfation at IdoA residues typically occurs at C2 position, and sulfation at GlcNS residues typically occurs at the C6 position. One or both residues can be sulfated in a given disaccharide unit. Ido(2S)-GlcNS(6S) disaccharide units make up about 85% of heparins from beef lung and about 75% of heparins from porcine intestinal mucosa. Heparin can also contain GlcA-GlcNAc and/or GlcA-GlcNS disaccharide units. Produced by basophils and mast cells, heparin acts as a naturally occurring anti-coagulant. The molecular weight of native heparin ranges from about 3 kDa to about 40 kDa, whereas the molecular weight of most commercially prepared heparin is about 12 kDa to about 15 kDa. A typical structure for the repeating disaccharide unit of which heparin is comprised is shown below:

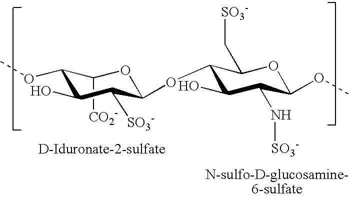

The term “hexose” is used herein to refer to a 6-carbon monosaccharide typically with an aldehyde (or potential aldehyde) at the C1 position (aldohexose) and hydroxyl groups at all other positions.

The term “hyaluronan” is used herein to refer to a glycosaminoglycan that is also known as “hyaluronic acid” and “hyaluronate” and is an unsulfated polymer of GlcA-GalNAc disaccharide units linked together via alternating 13-1,4 and β-1,3 glycosidic bonds. Hyaluronan is not found covalently linked to any protein. As many as 25,000 of these disaccharide units can make up a single hyaluronan polymer. In synovial fluid, the average molecular weight of hyaluronan is between three and four million daltons (Da), though hyaluronan can generally range in size from 5,000 to 20,000,000 Da in physiological environments. Hyaluronan is a major component of the extracellular matrix and is found in connective, epithelial, and neural tissues. It is one of the main components of synovial fluid, which lubricates joints. Hyaluronan also coats chondrocytes in cartilage and contributes to the resilient quality of cartilage. It is also abundant in the skin, where it plays a role in tissue repair. A typical structure for the repeating disaccharide unit of which hyaluronan is comprised is shown below:

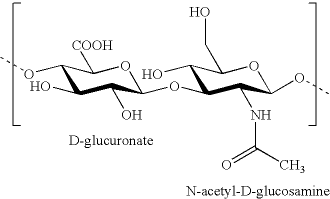

The term “hydrogen tetrachloroaurate” is used herein to refer to HAuCl₄, the product that forms when gold dissolves in aqua regia. Hydrogen tetrachloroaurate is also known as “gold chloride,” “chlorauric acid,” and “gold trichloride acid.” Aqua regia is a highly corrosive, fuming yellow or red solution that is formed by mixing concentrated nitric acid with concentrated hydrochloric acid, usually in a volumetric ratio of one to three respectively. Aqua regia is one of the few reagents that dissolves gold and platinum.

The abbreviation “IdoA” is used herein to refer to iduronic acid.

The abbreviation “IdoA(2S)” is used herein to refer to iduronic acid that is sulfated at the O₂ position.

The term “inflammation” is used herein to refer to a type of immune response leukocyte infiltration, redness, heat, pain, swelling, and/or often loss of function. Inflammation may serve to initiate the elimination of noxious agents and/or of damaged tissue. Generally, in inflammation, immune system components are directed to the site of injury or infection.

The terms “inflammatory disease” and “inflammatory condition” are used herein to refer to a disease or condition characterized by inflammation. Inflammatory diseases and conditions may be acute and/or chronic. Non-limiting examples of inflammatory diseases and conditions include acquired immune deficiency syndrome (AIDS), allograft rejection, adult respiratory distress syndrome, arthritis (including, for example, rheumatoid arthritis and osteoarthritis), asthma, atherosclerosis, autoimmune disorders (such as, for example, Addison's disease, autoimmune hepatitis Celiac disease, Crohn's Disease, giant cell arteritis, Goodpasture's syndrome, Grave's disease, Guillain-Barre syndrome, Hashimoto's thyroiditis, juvenile rheumatoid arthritis, lupus, polymyalgia rheumatica, psoriasis, primary biliary cirrhosis, rheumatoid arthritis, scleroderma, sclerosing cholangitis, Sjogren's syndrome, temporal arteritis, type 1 diabetes mellitus, ulcerative colitis, Wegener's granulomatosis, and combinations thereof), cancer, cerebral palsy, diabetes (including type 1 diabetes mellitus and type 2 diabetes mellitus), eczema, glomerulonephritis, heart failure, herpes dementia, immune complex diseases, infection caused by invasive microorganisms that produce nitric oxide (NO), inflammatory bowel disease, inflammatory sequelae of viral infections, ischemia (including ischemic brain edema), migraine, multiple sclerosis, myocarditis, organ transplant/bypass disorders, osteoporosis, oxidant induced lung injury, Paget's disease, pain, peritonitis, retinitis, sepsis and/or septic shock, sickle cell anemia, stroke, toxic shock syndrome, uveitus, X-adenoleukodystrophy (X-ALD), and combinations thereof. (It is noted that some of ordinary skill in the art would classify multiple sclerosis as an autoimmune disorder and others would not.) Many neurodegenerative diseases are also characterized by an inflammatory condition. Such neurodegenerative diseases include, for example, Alzheimer's disease, Parkinson's disease, Landry-Guillain-Barre-Strohl syndrome, multiple sclerosis, viral encephalitis, acquired immunodeficiency disease (AIDS)-related dementia, amyotrophic lateral sclerosis, brain trauma, and spinal cord disorders.

The term “in vitro” is used herein to refer to a controlled environment outside of a living organism, for example, in a plastic or glass dish that may be kept in an incubator. For example, experiments can be performed “in vitro,” meaning that biological material such as cells or molecules are manipulated experimentally in an environment outside of a living organism. In certain embodiments, the in vitro conditions are similar to physiological conditions (e.g., pH, osmolarity, etc.).

The term “keratan sulfate” is used herein to refer to a glycosaminoglycan also known as “keratosulfate” and comprised of sulfated Gal-GlcNAc disaccharide units linked together via alternating β-1,4 and β-1,3 glycosidic bonds and usually found covalently linked to a protein. The C6 atoms of GlcNAc and Gal residues are commonly sulfated in keratan sulfate. Keratan sulfate is found mostly in cornea, cartilage, and bone and acts as a cushion in joints. Several types of keratan sulfate have been delineated and classified according to structure of linkage to the core protein. Type I keratan sulfates are N-linked to asparagine residues in the core protein, and comprise at least two subtypes that are found in corneal tissue (“corneal keratan sulfate I”) and non-corneal tissues (“non-corneal keratan sulfate I”) such as cartilage and bone. Type II keratan sulfate is linked to protein via a GalNAc-β-serine/threonine linkage and found in cartilage. Type III keratan sulfate is linked to the core protein via a mannose-O-serine/threonine linkage and is found in proteoglycans from brain. The term “keratan” is used herein to refer to a glycosaminoglycan similar in structure to keratan sulfate, but lacking in sulfation in most or all residues. A typical structure for the repeating disaccharide unit of which keratan sulfate is comprised is shown below:

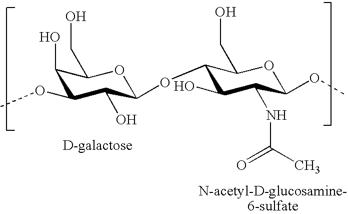

The term “lymph” is used herein to refer to a fluid that originates from body tissues that contains white blood cells and circulates through the body through the lymphatic system and is discharged into the bloodstream via the thoracic duct. Lymph acts to transport bacteria and protein from tissues, fat from small intestine, and lymphocytes to the blood.

The term “microparticle” is used herein to refer to a microscopic particle with at least one dimension between approximately 1 μm and approximately 1 mm in size. The term “microsphere” is used herein to refer to a spherical microparticle.

The term “monosaccharide” is used herein to refer to a carbohydrate that cannot be hydrolyzed into a simpler carbohydrate. Monosaccharides are the building blocks of oligosaccharides and polysaccharides. Examples include fructose, glucose, galactose, xylose, and ribose.

The term “nanoparticle” (used interchangeably with “nanocrystal” and also known in the art as “nanopowder” and “nanocluster”) is used herein to refer to a microscopic particle with at least one dimension less than 1 μm. In certain embodiments, at least one dimension of the microscopic particle is less than 100 nm. The phrase “metallic nanoparticle” refers to a nanoparticle comprising at least one metallic element.

The term “oligosaccharide” is used herein to refer to a linear or branched chain of typically less than 25 monosaccharides attached to one another via glycosidic linkages.

The term “pentose” is used herein to refer to a monosaccharide with five carbons, either having an aldehyde functional group at position 1 (aldopentose) or a ketone functional group in position 2 (ketopentose). Examples of typical aldopentoses include D-ribose, D-arabinose, D-xylose, and D-lyxose. Examples of typical ketopentoses include D-ribulose and D-xylulose. Pentoses may be cyclic or acyclic.

The term “plasma” is used herein to refer to blood plasma, the liquid component of blood that makes up about 55% of total blood volume. Blood plasma can be prepared, for example, by spinning blood in a centrifuge until the blood cells have gathered at the bottom of the tube, then collecting the fluid at the top (plasma).

The term “polysaccharide” is used herein to refer to a linear or branched chain of repeating monosaccharides, generally greater than 25 monosaccharide units in length and as many as thousands of monosaccharide units in length.

The phrase “polysaccharide lyase” is used herein to refer to a group of enzymes that cleave polysaccharide chains via a beta-elimination mechanism resulting in the formation of a double bond at the newly formed non-reducing end.

The term “proteoglycan” is used herein to refer to any protein with one or more covalently attached glycosaminoglycan chains. Proteoglycans are a major component of the extracellular matrix. Aside from forming a major structural component of connective tissue, proteoglycans are also involved in the movement of molecules through the extracellular matrix and in cellular signaling processes.

The phrase “reducing agent,” also known as a “reductant,” is used herein to refer to a chemical that can serve as an electron donor. As used herein, the phrase also encompasses solutions containing such a chemical or chemicals. For example, in certain embodiments of the invention, citrate solution is a reducing agent used to reduce Au³⁺ to Au⁰. Other reducing agents include, for example, sodium borohydride, thiocyanate, phosphorus, ascorbic acid, and ethyl alcohol.

The phrase “seminal fluid” also known as “semen,” is used herein to refer to a biological fluid that is secreted by the gonads and sexual organs of males and hermaphroditic animals and usually contains spermatozoa.

The term “serum” is used herein to refer to blood serum, the fluid that is obtained after having allowed blood to clot and taking the liquid portion. Serum is plasma without fibrinogen and other clotting factors.

The term “stereoisomer” is used herein to refer to a compound that has an identical chemical constitution, but differs in the arrangement of the atoms in space.

The term “stereogenic center” is used interchangeably with “chiral center” and is used herein to refer to an atom in a molecule that is bonded to four different chemical species, allowing for optical isomerism.

The term “sulfate” is used herein to refer to an SO₄⁻ group (in free solution), or an SO₃ group (as part of a compound), and is alternatively spelled “sulphate.” As part of a chemical name, the term “sulfate” refers to the chemical modified by the addition of a sulfate group. The term “sulfated” is used herein to refer mean “modified by the addition of a sulfate group,” and is alternatively spelled “sulphated”

The term “supernatant” is used herein to refer to any liquid above non-soluble solids and/or precipitates. The solids may be separated from the liquid supernatant by settling, sedimentation, precipitation, or centrifugation.

### DETAILED DESCRIPTION OF CERTAIN EMBODIMENTS OF THE INVENTION

The present invention provides metallic particles coated with glycosaminoglycans for use in detecting glycosaminoglycan degrading activity. Also provided are methods for preparing these particles, as well as methods of using these glycosaminoglycan-coated particles in biomedical and other applications. In certain embodiments, methods for assaying glycosaminoglycan-degrading activity using the glycosaminoglycan-coated particles are provided. Such methods may be used, for example, in diagnostic tests for diseases such as cancer and inflammatory diseases. The particles may also be used in assaying the activity of enzyme that degrades glycosaminoglycans.

I. Glycosaminoglycan-Coated Metallic Particles and their Synthesis

[symbol]

Provided particles typically comprise metal atoms. In certain embodiments, the particles comprise a metallic core. Any of a variety of metals may be used to prepare particles useful in the present invention. For example, in some embodiments of the invention, particles comprise gold atoms. In some embodiments of the invention, particles comprise cadmium atoms. In some embodiments of the invention, particles comprise lead atoms. Other metals that can be used to make particles of the present invention include silver, zinc, cobalt, iron, copper, manganese, nickel, titanium, palladium, platinum, etc. In some embodiments, particles comprise at least one metal and at least one other non-metallic element. In certain embodiments, the particles comprise a metallic salt. The salt may be an oxide, sulfide, or halide salt. In certain embodiments, the particles comprise a transition metal complex. For example, in certain embodiments of the invention, particles are comprised of cadmium and sulfur atoms. In certain embodiments of the invention, particles are comprised of lead and sulfur atoms. In certain embodiments of the invention, particles comprise more than one metal. For example, particles can comprise iron and cobalt atoms. In certain embodiments of the invention, particles comprise metal alloys. In certain embodiments, particles comprise non-metallic materials in addition to one or more metal(s). For example, particles may also comprise polymers, salts, organic compounds, etc., or combinations thereof.

Sizes of particles provided by the present invention may vary depending on the particular embodiment the particles are used for. In some embodiments of the invention, the particle is a nanoparticle, that is, having at least one dimension of its metallic portion less than approximately 1 μm. For example, the smallest dimension of the metallic portion of such particles can average between about 1 nm to about 10 nm. For example, the smallest dimension of the metallic portion of the particles can average about 1 nm, about 2 nm, about 3 nm, about 4 nm, about 5 nm, about 6 nm, about 7 nm, about 8 nm, about 9 nm, or about 10 nm. In some embodiments of the invention, the smallest dimension of the metallic portion of the particles averages between about 10 nm to about 30 nm. For example, the smallest dimension of the metallic portion of the particles can average about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, or about 30 nm. In some embodiments of the invention, the smallest dimension of the metallic portion of the particles averages between about 30 nm and about 100 nm. For example, the smallest dimension of [symbol] about 60 nm, about 70 nm, about 80 nm, about 90 nm, or about 100 nm. In some embodiments of the invention, the smallest dimension of the metallic portion of the particles averages between about 100 nm and about 1 μm. For example, the smallest dimension of the metallic portion of the particles can average about 100 nm, about 120 nm, about 140 nm, about 160 nm, about 180 nm, about 200 nm, about 220 nm, about 240 nm, about 260 nm, about 280 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1 μm. In some embodiments, the particle is approximately spherical in shape, and the smallest dimension of the metallic portion of the particle is the diameter of the metallic portion of the particle.

In some embodiments of the invention, the largest dimension of the metallic portion of the particles is less than about 1 μm. For example, the largest dimension of the metallic portion of the particles can fall between about 500 nm and about 1 μm, measuring about 500 nm, about 550 nm, about 600 nm, about 650 nm, about 700 nm, about 750 nm, about 800 nm, about 850 nm, about 900 nm, about 950 nm, or about 1 μm. The largest dimension of the metallic portion of the particles may fall between about 100 nm and about 500 nm, for example, about 100 nm, about 120 nm, about 140 nm, about 160 nm, about 180 nm, about 200 nm, about 220 nm, about 240 nm, about 260 nm, about 280 nm, about 300 nm, about 350 nm, about 400 nm, about 450 nm, or about 500 nm. In some embodiments of the invention, the largest dimension of the metallic portion of the particles falls between about 30 nm and about 100 nm, for example, about 30 nm, about 40 nm, about 50 nm, about 60 nm, about 70 nm, about 80 nm, about 90 nm, or about 100 nm. In some embodiments of the invention, the largest dimension of the metallic portion of the particles falls between about 10 nm and about 30 nm, for example, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, or about 30 nm. In some embodiments of the invention, the largest dimension metallic portion of the particles falls between about 1 nm and 10 nm, for example about 1 nm, about 2 nm, about 3 nm, about 4 nm, about 5 nm, about 6 nm, about 7 nm, about 8 nm, about 9 nm, or about 10 nm. In some embodiments, the particle is approximately spherical in shape, and the largest dimension of the metallic portion is the diameter of metallic portion of the particle.

In a given embodiment of the invention, the smallest dimensions of about 60%, particles fall within one standard deviation of the mean. Similarly, particle sizes in a given embodiment of the invention can vary such that the largest dimensions of about 60%, about 70%, about 80%, about 90%, or greater than about 90% of the metallic portions of the particles fall within one standard deviation of the mean.

Particles of the present invention can be any of a variety of shapes. For example, in some embodiments of the invention, particles are roughly spherical in shape. Additionally or alternatively, particles may be rod-shaped, cup-shaped, shell-shaped, cone shaped, pyramid-shaped, hexagonal cone-shaped, hexagonal-shaped, tetrahedral-shaped, cube-shaped, octahedral-shaped, dodecahedral-shaped, icosahedral-shaped, square-shaped, cross-shaped, V-shaped, plate-shaped, disc-shaped, spindle-shaped, ring-shaped, crown-shaped, shaped like a rice grain, peanut-shaped, etc. In some embodiments of the invention, the particles are irregular in shape. In certain embodiments, the particles are not consistently of one particular shape.

### Preparation of Metallic Particles

The present invention provides methods for preparing soluble metallic particles coated with glycosaminoglycans or other polysaccharides. Such methods involve using glycosaminoglycans or other polysaccharides. Without wishing to be bound by any particular theory, glycosaminoglycans or other polysaccharide present during early stages of the preparation of the particles may assist in the formation of soluble particles by binding to metal precursors and preventing the formation of a bulk semiconductor.

Generally, such methods comprise mixing a metal precursor (e.g., a metallic salt) with a glycosaminoglycan in an aqueous solution. In some embodiments, the glycosaminoglycan interacts with the metal precursor. The metal-glycosaminoglycan solution may in some embodiments be incubated (e.g., for a period of time at particular temperature, with or without stirring). A solution may be added to the metal-glycosaminoglycan solution and may provide another precursor (such as a source of sulfide) that will be a part of the synthesized metallic particle, or it may facilitate reducing the metallic salt to a metal, or both. Typically, further purification steps are not necessary, though in certain embodiments of the invention, methods comprise isolating, purifying, and/or sizing the glycosaminoglycan-coated metallic particles. In certain embodiments, the particles are sized (e.g., using a sieve or filter).

In certain embodiments of the invention, glycosaminoglycan-coated gold [symbol] containing compound) with a glycosaminoglycan or other polysaccharide in an aqueous solution, heating the resulting solution to a temperature greater than approximately 40° C., and adding a reducing agent to the stirred gold-glycosaminoglycan solution. In some embodiments, the glycosaminoglycan interacts with the gold salt during the mixing. In certain embodiments of the invention, the reducing agent reduces Au³⁺ to Au⁰. Any agent that has the potential to reduce gold could be used in this procedure. Examples of reducing agents include citrate salts or citric acid, sodium borohydride, thiocyanates, ascorbic acid, ethyl alcohol, etc. In certain embodiments, the reducing agent is a citrate salt.

In certain embodiments, the concentration of reducing agent in the reaction is between approximately 0.01% and approximately 10% (weight/volume). In certain embodiments of the invention, the concentration of reducing agent in the reaction is between approximately 0.02% and 8% (weight/volume). In certain embodiments of the invention, the concentration of reducing agent in the reaction is between approximately 0.05% and approximately 0.5% (weight/volume). In certain embodiments of the invention, the concentration of reducing agent in the reaction is approximately 0.02% (weight/volume). In certain embodiments of the invention, the concentration of reducing agent in the reaction is approximately 0.5% (weight/volume).

In certain embodiments of the invention, all steps in the synthesis reaction are carried out at temperatures under approximately 80° C. In certain embodiments of the invention, steps in the synthesis reaction are carried out at temperatures ranging between approximately 70° C. and approximately 80° C., between approximately 60° C. and approximately 70° C., between approximately 50° C. and approximately 60° C., between approximately 40° C. and approximately 50° C., between approximately 30° C. and approximately 40° C., or between approximately 20° C. and approximately 30° C. In certain embodiments of the invention, the glycosaminoglycan-coated gold particles are soluble in water.

In certain embodiments of the invention, cadmium sulfide particles are prepared. For example, Cd(NO₃)₂ or another cadmium-containing compound (e.g., a cadmium salt) can be first mixed with a glycosaminoglycan or other polysaccharide. Sodium sulfide or other source of sulfide anion can then be added to the resulting solution. In certain embodiments of the invention, all steps in the synthesis reaction are carried out at temperatures under approximately 80° C. In certain embodiments of the invention, steps in the synthesis reaction are carried out at a temperature ranging between approximately 70° C. approximately 50° C. and approximately 60° C., between approximately 40° C. and approximately 50° C., between approximately 30° C. and approximately 40° C., or between approximately 20° C. and approximately 30° C. In certain embodiments, such a synthetic scheme yields water-soluble cadmium sulfide particles. In some such embodiments, the synthesized cadmium sulfide particles are coated with glycosaminoglycans.

In certain embodiments of the invention, lead sulfide particles are prepared. For example, Pb(NO₃)₂ or another lead-containing compound (e.g., a lead salt) is mixed with a glycosaminoglycan or other polysaccharide, and the resulting solution incubated at room temperature for a period of time before the addition of a source of sulfide anions (e.g., sodium sulfide). In certain embodiments of the invention, all steps in the synthesis reaction are carried out at temperatures under approximately 80° C. In certain embodiments of the invention, steps in the synthesis reaction are carried out at a temperature ranging between approximately 70° C. and approximately 80° C., between approximately 60° C. and approximately 70° C., between approximately 50° C. and approximately 60° C., between approximately 40° C. and approximately 50° C., between approximately 30° C. and approximately 40° C., or between approximately 20° C. and approximately 30° C. In certain embodiments, such a procedure yields water-soluble lead sulfide particles. In some such embodiments of the invention, the synthesized lead sulfide particles are coated with glycosaminoglycans.

In certain embodiments of the invention, the metal precursor is mixed with more than one type of glycosaminoglycan or polysaccharide. In some such embodiments, the resulting particles include particles each having more than one type of glycosaminoglycan on its surface. Alternatively or additionally, the resulting particles include particles that are each coated with only one type of glycosaminoglycan, but different particles in the population may be coated with different glycosaminoglycans.

### Glycosaminoglycans

Particles can be coated with one or more of glycosaminoglycans, that is, a linear unbranched polysaccharide chain comprising a repeating disaccharide unit, which typically comprises hexosamine and a hexose or a hexuronic acid linked together by a glycosidic bond. In certain embodiments, the disaccharide unit of the glycosaminoglycan includes a glucosamine or galactosamine moiety followed by a uronic acid moiety. The glycosaminoglycan may be naturally occurring or synthetically produced. Glycosaminoglycans form a major component of the extracellular matrix and of glycoproteins typically found on the surface of cells, and GAGs are the most abundant heteropolysaccharides in the body. Often they are attached to proteins to form proteoglycans. Their diverse functions in the cell include, for example, providing structural rigidity that is needed for cell migration, contributing to the viscosity of the fluid of which they are a part, and ligand-receptor recognition.

For example, in some embodiments of the invention, the glycosaminoglycan is heparan sulfate composed of at least 20%, at least about 30%, at least about 40%, at least about 50%, at least about 60%, at least about 70%, at least about 80%, or more of GlcA-GlcNAc disaccharide units linked together via alternating α-1,4 glycosidic bonds. Heparan sulfate is typically further comprised of IdoA-GlcN disaccharide units. The IdoA-GlcN disaccharide units can be sulfated, for example, at the C2 position of the IdoA residue, at the C6 position of the GlcN residue in both residues, or at neither position of a given disaccharide unit. The glycosaminoglycan can be heparan sulfate that is mono- or di-sulfated in at least 10%, at least about 20%, at least about 30%, at least about 40%, at least about 50%, at least about 60%, at least about 70%, at least about 80%, at least about 90%, or more of the disaccharide units. The glycosaminoglycan can be a heparan sulfate comprising other saccharide units such as 3-O-sulfated glucosamine (GlcNS(3S,6S) and/or a free amine group (GlcNH₃⁺).

In some embodiments of the invention, the glycosaminoglycan is heparin composed of at least 40%, at least about 50%, at least about 60%, at least about 70%, at least about 80%, at least about 90%, or more IdoA(2S)-GlcNS(6S) disaccharide units.

In some embodiments of the invention, the glycosaminoglycan is chondroitin sulfate. The glycosaminoglycan can be, for example, chondroitin sulfate that is sulfated at the C6 position of the GalNAc residues, also known as chondroitin sulfate A and chondroitin 6-sulfate. The glycosaminoglycan can be chondroitin sulfate that is sulfated at the C4 position of the GalNAc residues, also known as chondroitin sulfate C and chondroitin 4-sulfate. The glycosaminoglycan can be chondroitin sulfate that is sulfated at the C2 position of the GlcA residues as well as the C6 position of the GalNAc residues, also known as chondroitin sulfate D and chondroitin-2,6-sulfate. The glycosaminoglycan can be chondroitin sulfate that is sulfated on the C4 and C6 positions of the GalNac residues, also known as chondroitin sulfate E and chondroitin-4,6,-sulfate. Sulfation at any or a combination of the above positions can occur in at least 10%, at least about 20%, at least [symbol] least about 80%, at least about 90%, or more of the disaccharide units.

In some embodiments of the invention, the glycosaminoglycan is chondroitin sulfate containing a mixture of residues typical of any of the above-mentioned subtypes of chondroitin sulfate. For example, some disaccharide units in the chondroitin sulfate may be unsulfated, others may be sulfated once, and yet others may be sulfated twice. In some embodiments of the invention, the glycosaminoglycan is a chondroitin GAG that is all or mostly unsulfated, referred to as chondroitin.

In some embodiments of the invention, the glycosaminoglycan is dermatan sulfate (also know has chondroitin sulfate B). The extent of sulfation along the polysaccharide may vary in different versions of the GAG. The C4 position of GalNAc residues can be sulfated in at least 10%, at least about 20%, at least about 30%, at least about 40%, at least about 50%, at least about 60%, at least about 70%, at least about 80%, at least about 90%, or more of the disaccharide units. In some embodiments of the invention, the glycosaminoglycan is a dermatan chain that is sulfated in less than about 10% of the disaccharide units, referred to as dermatan.

In some embodiments of the invention, the glycosaminoglycan used in accordance with the invention is keratan sulfate. The glycosaminoglycan can be, for example, any subtype of keratan sulfate, including but not limited to type I keratan sulfate (both corneal and non-corneal), type II keratan sulfate, and type III keratan sulfate. Gal-GlcNAc disaccharide units within keratan sulfate can be sulfated, for example, at the C6 position of the Gal residue, at the C6 position of the GlcNAc residue, at both positions, or at neither position. The glycosaminoglycan can be, for example, keratan sulfate that is mono- or di-sulfated in at least 10%, at least about 20%, at least about 30%, at least about 40%, at least about 50%, at least about 60%, at least about 70%, at least about 80%, at least about 90%, or more of the disaccharide units. In some embodiments of the invention, the glycosaminoglycan is a keratan that is sulfated in less than about 10% of the disaccharide units, referred to herein as keratan.

In some embodiments of the invention, the glycosaminoglycan is hyaluronan (also known as hyaluronic acid and hyaluronate).

In some embodiments of the invention, more than one of the above mentioned types or variants of glycosaminoglycans described above are attached to a single particle. For example, glycosaminoglycan molecules having different extents of sulfation along the polysaccharide chains may be attached to a single particle. Alternatively or additionally, [symbol] attached to a single particle. In some embodiments of the invention, a population of particles each coated with a particular kind of glycosaminoglycan, but not necessarily the same kind of glycosaminoglycan coating the other particles in the population, are used together in a single assay for glycosaminoglycan-degrading activity.

One of ordinary skill in the art will appreciate that the nomenclature of glycosaminoglycans is sometimes complex. For example, the difference between chondroitin sulfate and dermatan sulfate is epimerisation of glucoronic acid to iduronic acid. A chondroitin sulfate chain may have dermatan sulfate residues within the chain. Depending on the extent of sequences of dermatan sulfate within the chain, it may be difficult to classify the molecule as either chondroitin sulfate or dermatan sulfate, though the molecule is clearly recognizable as a glycosaminoglycan. Similarly, heparin and heparan sulfate are closely related, and heparin is sometimes classified as a variant of heparan sulfate. Also, hybrid molecules exist that comprise both “heparin-like” and “heparan sulfate-like” residues. It can be appreciated that the provided methods can also be used with such molecules that are not readily classified as being of one particular type of glycosaminoglycan. In certain embodiments, provided are particles coated with such glycosaminoglycans.

Similarly, the glycosaminoglycan can be a non-naturally occurring glycosaminoglycan. For example, chimeric and/or hybrid glycosaminoglycans can be synthesized as described in published US patent application, US 2006/0188966, incorporated herein by reference in its entirety. The disaccharide units themselves can be synthetic disaccharides that are not known to exist in naturally occurring glycosaminoglycans. For example, some synthetic disaccharides in glycosaminoglycans can comprise at least one pentose sugar. Such pentose sugars include, but are not limited to, ribose, arabinose, xylose, lyxose, ribulose, and xylulose. Disaccharide units may also include non-natural saccharides.

A glycosaminoglycan typically comprises many disaccharide units. In some embodiments, a glycosaminoglycan comprises more than 2, more than 3, more than 4, more than 5, more than 10, more than 15, more than 25, more than 50, more than 100, more than 200, more than 500, more than 1000, or more disaccharide units in a single molecule.

In some embodiments, glycosaminoglycans are attached to metal atoms in the metallic particle in a particular way, e.g., with a particular chemistry. Without wishing to be bound by any particular theory, it is proposed that the biological activity of a glycosaminoglycan may be lost if reactive groups on carbohydrate residues within the glycosaminoglycan are lost. In some embodiments, the glycosaminoglycan is not attached to [symbol] activity of the glycosaminoglycan is not destroyed by its attachment to the particle. In certain embodiments, the glycosaminoglycan is attached to the particle via a covalent bond. In certain embodiments, the glycosaminoglycan is attached to the particle via a non-covalent interaction.

### II. Assaying the Activity of a Glycosaminoglycan-Degrading Enzyme

### Assays

Provided are methods for assaying glycosaminoglycan-degrading activity using metallic particles coated with a glycosaminoglycan. Generally, such methods involve providing a metallic particle coated with a glycosaminoglycan that is capable of being degraded by an enzyme that is being assayed for, contacting such particles with a sample under suitable conditions to allow enzymes in the sample to degrade the glycosaminoglycans on the surface of the particles, and determining the activity of the glycosaminoglycan-degrading enzyme. Degradation of the glycosaminoglycan coating the metallic particle typically leads to aggregation of the metallic particles, and detection of such aggregation (e.g., by spectroscopic methods) forms the basis of determining the presence of glycosaminoglycan-degrading activity in the sample being tested.

### Glycosaminoglycan-Degrading Enzymes

Glycosaminoglycan-degrading activity that can be assayed for using the inventive methods may, for example, be mediated by an enzyme. These enzymes are typically broadly classified as polysaccharide lyases or as glycoside hydrolases. In certain embodiments, the enzyme is a non-specific enzyme capable of degrading a GAG.

In some embodiments of the invention, the glycosaminoglycan-degrading activity being assayed is mediated by a polysaccharide lyase. Examples of polysaccharide lyases include, but are not limited to, pectate lyase, pectin lyase, exo-pectate lyase, exo-polygalacturonate lyase, rhamnogalacturonan lyase, alginate lyase, chondroitinase B, α-L-guluronate lyase, hyaluronate lyase; chondroitin ABC lyase, chondroitin AC lyase, xanthan lyase, exopolygalacturonate lyase, heparin-sulfate lyase, heparin lyase, polysaccharide lyase acting on glucuronic acid, oligo-alginate lyase, and hyaluronan lyase. More information about these polysaccharide lyases can be found in the CAZy database (database of carbohydrate-active enzymes) at www.cazy.org/fam/acc_PL.html.

In certain embodiments of the invention, the glycosaminoglycan-degrading proteins in the glycoside hydrolases family include α-N-acetylgalactosaminidase, α-N-acetylglucosaminidase, β-N-acetylhexosaminidase, β-1,4-N-acetylmuramoylhydrolase, α-agarase, β-agarase, alkaline and neutral invertase, alternansucrase, amygdalin b-glucosidase, α-amylase, β-amylase, amylomaltase or 4-a-glucanotransferase, amylopullulanase, amylosucrase, arabinanase, α-L-arabinofuranosidase, branching enzyme, κ-carrageenase, λ-carrageenase, cellobiohydrolase, cellobiohydrolases, cellobiose phosphorylase, cellodextrin phosphorylase, cellulase, cellulose 1,4-b-cellobiosidase, chitinase, chitobiose phosphorylase, chitosanase, concanavalin B, cyclic β-1,2-glucan synthase, cycloisomaltooligosaccharide glucanotransferase, cyclomaltodextrin glucanotransferase, cyclomaltodextrinase, dextran 1,6-α-isomaltotriosidase, dextranase, dextransucrase, endo-1,3(4)-β-glucanase, endo-1,3-β-xylanase, endo-1,3-glucanase, endo-1,4-β-galactanase, endo-1,4-β-xylanase, endo-1,6-β-galactanase, endo-α-N-acetylgalactosaminidase, endo-β-galactosidase, endo-β-glucuronidase/heparanase, endo-β-N-acetylglucosaminidase, endoglucanase, endo-N-acetylneuraminidase, endo-processive cellulases, endorhamnosidases, endo-sialidase, endo-xylogalacturonan hydrolase, exo-1,3-1,4-glucanase, exo-1,3-glucanase, exo-arabinanase, exo-β-glucosaminidase, exo-inulinase, exo-polygalacturonase, exo-polygalacturonosidase, fructan β-(2,1)-fructosidase, fructan fructan 1-fructosyltransferase, fructan β-(2,6)-fructosidase, 2,6-β-fructan 6-levanbiohydrolase, β-fructofuranosidase, α-1,2-L-fucosidase, α-L-fucosidase, β-D-fucosidase, galactan 1,3-β-galactosidase, galactocerebrosidase, α-galactosidase, β-galactosidase, glucan 1,3-β-glucosidase, glucan 1,4-β-glucosidase, glucan endo-1,3-β-D-glucosidase, glucan endo-1,613-glucosidase, β-1,3-glucan transglycosidases, α-glucan lyase, α-1,3-glucanase, β-1,3-glucanase, β-1,6-glucanase, β-1,3-glucanosyltransglycosylase, 4-α-glucanotransferase, glucoamylase, glucodextranase, α-glucosidase, α-1,3-glucosidase, glucosylceramidase, α-glucuronidase, β-glucuronidase, glycoprotein endo-α-1,2-mannosidase, β-glycosidase, hemagglutinin-neuraminidase, β-hexosaminidase, hyaluronidase, hydroxyisourate hydrolase, α-L-iduronidase, inulin fructotransferase (depolymerizing, difructofuranose-1,2′:2,3′-dianhydride-forming), inulinase, inulosucrase, invertase, isoamylase, isoflavonoid 7-O-β-apiosyl-β-glucosidase, isomalto-dextranase, isomaltosyltransferase, isomaltulose synthase, isopullulanase, keratan sulfate hydrolase (endo-β-N-acetylglucosaminidase), keratan-sulfate endo-1,4-β-galactosidase, kojibiose phosphorylase, α-lactalbumins, lactase, lacto-N-biosidase, levanase, levansucrase, licheninase, lysozyme, lysozyme type C, lysozyme type G, lysozyme type i, maltogenic maltopentaose-forming α-amylase, maltose phosphorylase, maltose-6-phosphate glucosidase, maltotetraose-forming α-amylase, mannan endo-1,4-β-mannosidase, β-1,3-mannanase, mannanase, α-1,6-mannanase, α-mannosidase, α-1,2-mannosidase, β-mannosidase, mannosylglycoprotein endo-β-mannosidase, mycodextranase, N-acetyl β-glucosaminidase, N-acetylmuramidase, narbonin, neopullulanase, neuraminidase, oligo-α-glucosidase, oligoxyloglucan reducing end-specific cellobiohydrolase, peptidoglycan hydrolases with endo-β-N-acetylglucosaminidase specificity, peptidoglycan lytic transglycosylase, phlorizin hydrolase, 6-phospho-β-galactosidase, 6-phospho-β-glucosidase, polygalacturonase, β-primeverosidase, processing α-glucosidase, prunasin β-glucosidase, pullulanase, raffinose synthase, raucaffricine β-glucosidase, reducing end-acting cellobiohydrolase, reducing-end-xylose releasing exo-oligoxylanase, rhamnogalacturonan, α-L-rhamnopyranohydrolase, rhamnogalacturonase, α-L-rhamnosidase, sialidase, stachyose synthase, strictosidine 13-glucosidase, sucrase-isomaltase, sucrose phosphorylase, sucrose:sucrose 1-fructosyl transferase, sulfated fucan endo-1,4-fucanase, thioglucosidase, trans-sialidase, α,α-trehalase, trehalase, trehalose phosphorylase, trehalose synthase, trehalose-6-phosphate hydrolase, d-4,5 unsaturated β-glucuronyl hydrolase, unsaturated rhamnogalacturonyl hydrolase, xylan 1,4-β-xylosidase, xylan α-1,2-glucuronosidase, xylanase, β-1,3-xylanase, xylanase inhibitors, xyloglucan endotransglycosylase, xyloglucan hydrolase, xyloglucan:xyloglucosyl transferase, xyloglucanase, xyloglucan-specific endo-β-1,4-glucanase, α-xylosidase, and β-xylosidase. More information about these glycoside hydrolases can be found in the CAZy database at www.cazy.org/fam/acc_GH.html.

Glycosaminoglycan-degrading activity can be mediated, for example, by an enzyme that is a member of an enzyme family known to degrade glycosaminoglycans. Exemplary families include the heparanases, the heparinases, the hyaluronidases, the chondroitinases, the dermatanases, and the keratanases. Activity of enzymes from other families that degrade glycosaminoglycans, including those not yet characterized and/or discovered, may also or alternatively be assayed.

### Heparanase/Heparinase

In certain embodiments of the invention, the glycosaminoglycan-degrading enzyme being assayed is a heparanase, which degrades heparan sulfate. For example, the activity of heparanases can be assayed using heparan sulfate-coated particles. The [symbol] as CTAPIII, a 9-10 k Da protein derived from the CXC chemokine, platelet basic protein; Hpa1 heparanase, a 45-50 kDa glycoprotein found in placenta and platelets (and which, as a higher molecular weight aggregate, is identical to the originally discovered 130 kDa heparanase derived from platelets); the three predicted protein products of the Hpa2 heparanase gene, which vary in size from 48 to 60 kDa; Chinese Hamster Ovary (CHO) cell heparanase (comprised of four fractions: CIA heparanase, a 37-48 kDa protein; C1B heparanase, a 30 kDa protein; C2A heparanase, a 45 kDa protein; and C2B heparanase, a 45 kDa protein); and tumor-derived heparanases such as the 96 kDa melanoma heparanase. See, e.g., Glycobiology. 2001 June; 11(6):91R-98R for discussion of these heparanases.

Heparanases such as, for example, CTAPIII, Hpa1, Hpa2, and CHO heparanases may be overexpressed in tumor cells. For example, Hpa1 heparanase has been isolated from human hepatoma cells. Thus, some tumor-derived heparanases are identical to one of the other heparanases described above. The heparanases mentioned above are classified as endo-β-glucuronidases based on their chemical specificity.

In some embodiments of the invention, the glycosaminoglycan-degrading enzyme being assayed for is a heparinase. These include, but are not limited to, heparinases derived from the organism Flavobacterium heparinum: heparinase I (HepI), a 43 kDa enzyme that degrades both heparin and heparan sulfate; heparinase II (HepII), an 85 kDa enzyme that degrades mainly heparin; and heparinase III (HepIII), a 71 kDa enzyme that degrades mainly heparan sulfate.

### Hyaluronidase

In some embodiments of the invention, the glycosaminoglycan-degrading enzyme being assayed for is a hyaluronidase (also known as hyaluronate lyase and hyaluronan lyase), which degrades hyaluronan. For example, hyaluronidase activity can be assayed using hyaluronan-coated particles provided by the present invention. A family of enzymes known as the hyaluronidases (also know as “hyases”) are known to degrade hyaluronan into low molecular weight hyaluronan and oligosaccharides. Hyaluronidases have been classified into three major groups according to their enzymatic mechanisms. Endo-β-N-acetyl-hexosaminidases constitute two of those groups. One such group includes vertebrate enzymes that use substrate hydrolysis. The other such group includes mostly bacterial enzymes that function by β-elimination of the glycosidic linkage with the introduction of an unsaturated bond. These predominantly bacterial enzymes are referred to as “eliminases” as leeches and some crustaceans.

Eukaryotic hyaluronidases are classified as glycoside hydrolase family 56 in the CAZy database (www.cazy.org), a database of carbohydrate-active enzymes. In some embodiments of the invention, the glycosaminoglycan-degrading enzyme is a human hyaluronidase or hyaluronidase-like enzyme, such as hyaluronidase 2, hyaluronidase 3, hyaluronoglucosaminidase 4, hyaluronidase 1, hyaluronidase, and ENSP00000194108. In some embodiments of the invention, the glycosaminoglycan-degrading enzyme is a mammalian hyaluronidase.

In certain embodiments of the invention, the glycosaminoglycan-degrading enzyme being assayed for is an enzyme in the same glycoside hydrolase family, but derived from non-human species. These include, but are not limited to, enzymes and similar gene products from Mus musculus (hyaluronidase, hyaluronidase 2, Hyal4, hyaluronidase 3, the protein corresponding to RIKEN cDNA clone 4932701A20 or 4930503G08, Hyal-5, and hyaluronoglucosaminidase 1), Rattus norvegicus (hyaluronidase 3 (Hyal3), hyaluronidase, hyaluronidase 1 (Hyal1), the protein corresponding to Mammalian Gene Collection clone MGC:9429=3 (See http://mgc.nci.nih.gov/), the protein corresponding to Mammalian Gene Collection clone MGC:108951, hyaluronidase 4 (Hyal4), and 2B1 antigen), Danio rerio (the protein corresponding to Mammalian Gene Collection clone MGC:158668, the protein corresponding to Mammalian Gene Collection clone MGC:136804, the protein corresponding to Sanger Institute annotated genomic clone DKEY-4219.14-001, the protein corresponding to MGC:158626, the protein corresponding to NCBI Accession Number AAI29331.1, the protein corresponding to Sanger Institute annotated genomic clone DKEY-72L14.3-001), Xenopus laevis (lysosomal hyaluronidase (Hyal2), embryonic hyaluronidase XEH1, and hyaluronidase XKH1), Xenopus tropicalis (the protein corresponding to MGC:121214), and Caenorhabditis elegans (the protein corresponding to C. elegans gene name T22C8.2).

A list of these and other eukaryotic hyaluronidases and their GenBank/GenPept and UniProt accession numbers can be found at www.cazy.org/fam/GH56.html, which is incorporated herein by reference in its entirety. In the above list of hyaluronidase enzymes, where genes or protein products have not been fully characterized, identifying numbers have been provided that can be matched to the list at www.cazy.org/fam/GH56.html.

In some embodiments of the invention, the glycosaminoglycan-degrading enzyme being assayed for is hyaluronidase present in and/or derived from insect tissues or venom. For example, hyaluronidases have been detected in the venom of snakes, fish, bees, wasps, scorpions, spiders, etc. The activity of such hyaluronidases can be assayed using particles provided by the present invention.

In some embodiments of the invention, the glycosaminoglycan-degrading enzyme is hyaluronidase produced by and/or derived from bacterial and other prokaryotic organisms. For example, pathogenic Gram-positive bacteria produce hyaluronidases, and such hyaluronidases may be involved in the disease process. Assays of such activity may aid in diagnosing an infection or following the progress of treatment.

It should be noted that although the enzymes listed above are classified as hyaluronidases, many are known to also have limited ability to degrade other glycosaminoglycans, namely chondroitin and chondroitin sulfates. In some embodiments of the invention, chondroitin- or chondroitin sulfate-coated particles, or particles coated with a mixture of glycosaminoglycans including all or a subset of chondroitin, chondroitin sulfate, and hyaluronan, are used to assay the activity of hyaluronidases. In some embodiments of the invention, a population of particles each coated with different glycosaminoglycans including all or a subset of chondroitin, chondroitin sulfate, and hyaluronan, are used to assay hyaluronidase activity.

### Chondroitinase

In certain embodiments of the invention, the glycosaminoglycan-degrading enzyme being assayed for is a chondroitinase, also known as chondroitin sulfate lyase and chondroitin sulfate eliminase. Examples of chondroitinases include chondroitinase AC-I from Flavobacterium heparinum (ChnAC, EC 4.2.2.4, also known as chondroitin AC lyase), a 75 kDa enzyme that degrades chondroitin sulfate A, chondroitin sulfate C, and hyaluronan; chondroitinase AC-II from Arthrobacter aurescens (EC 4.2.2.5), a 76 kDa enzyme that degrades chondroitin sulfate A, chondroitin sulfate C, and hyaluronan; chondroitinase B from Flavobacterium heparinum (ChnB, no EC number, also known as dermatanase, dermatan sulfate eliminase, and dermatan sulfate lyase), a 55 kDa enzyme that degrades dermatan sulfate; and chondroitinase ABC from Proteus vulgaris (EC 4.2.2.4, also known as chondroitinase ABC lyase and chondroitinase ABC eliminase), a 120-145 kDa enzyme that degrades chondroitin sulfate A, chondroitin sulfate C, dermatan sulfate, and hyaluronan.

It should be noted, as mentioned above, that some of the enzymes classified as chondroitinases degrade dermatan sulfate, and some also degrade hyaluronan. In some embodiments of the invention, particles coated with chondroitin, chondroitin sulfate, some embodiments of the invention, particles coated with a mixture of glycosaminoglycans including all or a subset of chondroitin, chondroitin sulfate, dermatan, dermatan sulfate, and hyaluronan, are used to assay chondroitinase activity. In some embodiments of the invention, a population of particles each coated with different glycosaminoglycans including all or a subset of chondroitin, chondroitin sulfate, dermatan, dermatan sulfate, and hyaluronan, are used to assay hyaluronidase activity. In certain embodiments of the invention, more than one type of glycosaminoglycan is coated on an metallic particle. For example, in some embodiments, at least two of the glycosaminoglycans selected from the group consisting of chondroitin, chondroitin sulfate, dermatan, dermatan sulfate, and hyaluronan are coated on the same metallic particles. In certain embodiments of the invention, at least three of the glycosaminoglycans selected from the group consisting of chondroitin, chondroitin sulfate, dermatan, dermatan sulfate, and hyaluronan are coated on the same inventive metallic particles. In certain some embodiments of the invention, at least four of the glycosaminoglycans selected from the group consisting of chondroitin, chondroitin sulfate, dermatan, dermatan sulfate, and hyaluronan are coated on the same metallic particles. In certain embodiments of the invention, all five of the glycosaminoglycans selected from the group consisting of chondroitin, chondroitin sulfate, dermatan, dermatan sulfate, and hyaluronan are coated on the same metallic particles.

In certain embodiments, particles are each coated with one particular glycosaminoglycan, but used together in an assay with other particles each coated with a different glycosaminoglycan. For example, more than one of particles selected from the group consisting of chondroitin-coated particles, chondroitin sulfate-coated particles, dermatan-coated particles, dermatan sulfate-coated particles, and hyaluronan-coated particles.

### Other Glycosaminoglycan-Degrading Enzymes

In certain embodiments of the invention, the glycosaminoglycan-degrading enzyme being assayed is a keratanase, which degrades keratan and/or keratan sulfate. An example of a keratanase is an enzyme derived from Pseudomonas also known as keratan sulfate endo-β-galactosidase.

The activity of other endo-D-glucuronidases, including non-mammalian and recombinant endo-D-glucuronidases, can also be assayed using glycosaminoglycan-coated particles of the present invention.

Glycosaminoglycan-degrading enzymes that can be assayed using the methods of the present invention may be obtained from natural sources (e.g., from organisms such as those mentioned above, from cells grown in culture, from cell lysates, from biological fluids, etc), chemically synthesized, produced by recombinant DNA technology, etc. For example, purified enzymes from recombinant sources may be used in positive controls for the inventive methods. Glycosaminoglycan-degrading enzymes can be assayed regardless of their source of origin so long as they are able to degrade glycosaminoglycans.

### Samples

In various embodiments, the invention provides methods for assaying the glycosaminoglycan-degrading activity in a variety of samples. Samples may be obtained from a variety of sources including, but not limited to, bioreactors, biological samples, and/or biological materials involved in the production of a therapeutic and/or commercial product (e.g., recombinant enzyme). For example, provided methods may be used to assay glycosaminoglycan-degrading activity in a therapeutic and/or commercial product. In some embodiments, glycosaminoglycan-degrading activity is monitored during production of the therapeutic and/or commercial product.

In some embodiments, glycosaminoglycan-degrading activity is desirable in a product, therapeutic formulation, etc. In some such embodiments, the product or therapeutic formulation comprises a glycosaminoglycan-degrading enzyme, and provided methods are used to monitor production and/or activity of the enzyme.

In some embodiments, glycosaminoglycan-degrading activity is not desirable in a product, therapeutic formulation, etc. In some such embodiments, glycosaminoglycan-degrading activity is monitored during production to facilitate selection of production batches that do not contain more glycosaminoglycan-degrading activity than is desirable in the product.

In some embodiments, enzyme activity is measured in cells or samples obtained from cells, such as cells being grown in cultures. Cells may have altered expression of one or more glycosaminoglycan-degrading enzymes. Such altered expression may be induced intentionally (such as, for example, by genetic engineering to overexpress, mutate, inactivate, etc. a gene encoding an enzyme that degrades glycosaminoglycans), or may not be (such as, for example, in disease states, by naturally occurring mutations, etc.).

In some embodiments, the sample being assayed is a biological sample. In some [symbol] from whom the biological sample is collected can be a human, such as a patient suffering from a disease or a healthy individual. In certain embodiments of the invention, the individual is suffering from cancer or is at risk of developing cancer. In certain embodiments of the invention, the individual has multiple myeloma or is at risk of developing multiple myeloma. In certain embodiments of the invention, the individual has prostate cancer or is at risk of developing prostate cancer. In certain embodiments of the invention, the individual is suffering from an inflammatory disease, is suspected of having an inflammatory disease, or is at risk for developing an inflammatory disease. In certain embodiments of the invention, the individual is suffering from an autoimmune disease, is suspected of having an autoimmune disease, or is at risk for developing an autoimmune disease. Examples of autoimmune diseases include, but are not limited to, rheumatoid arthritis, diabetes mellitus type 1, systemic lupus erythrematosis, Sjögren's disease, multiple sclerosis, Hashimoto's thyroiditis, Grave's disease, Coeliac disease, and idiopathic thrombocytopenic purpura. In certain embodiments of the invention, the individual has an infectious disease, is suspected of having an infectious disease, or is at risk for having an infectious disease. In certain embodiments of the invention, the individual is infected with a pathogenic agent, is suspected of having been infected with a pathogenic agent, or is at risk for being infected with a pathogenic agent.

In some embodiments of the invention, the biological sample is collected from an animal. For example, it may be desirable to assay the activity of glycosaminoglycan-degrading enzymes in biological samples from animals used in biomedical research (such as disease models, genetically modified organisms, animals in which antibodies are raised or biological materials are extracted, etc.) or commercially relevant animals (such as farm animals). These animals include, but are not limited to, non-human primates (such as rhesus macaques, pig-tailed macaques, cynomolgus macaques, owl monkeys, capuchin monkeys, squirrel monkeys, tamarins, common marmosets, chimpanzees, etc.), rodents (such as rats, mice, hamsters, etc.), guinea pigs, amphibians (such as frogs, etc.), reptiles, fish (such as zebrafish, Medaka, pufferfish, etc.), birds (such as chickens, zebrafinches (songbirds), pigeons, etc.), dogs, rabbits, horses, donkeys, goats, cattle, pigs, sheep, chickens, etc. Other organisms from which biological samples can be prepared that can be used with some embodiments of the present invention include, without limitation, bacteria, yeasts, nematode worms (such as Caenorhabditis elegans, etc.), fruit flies (such as Drosophila, etc.), plants (such as Arabidopsis, tobacco, maize, etc.), etc.

The biological sample can be a fluid such as serum, plasma, blood, lymph, ascites fluid, urine, saliva, synovial fluid, cerebrospinal fluid, tear fluid, vitreous humor, venom, seminal fluid, cell supernatant, cell lysate, or cell culture medium.

In certain embodiments of the invention, the biological sample is blood or a blood component or related fluid such as serum, plasma, or lymph. In certain embodiments of the invention, the biological sample is urine. It may be desirable to assay for glycosaminoglycan-degrading activity in such bodily fluids, as both glycosaminoglycans and glycosaminoglycan-degrading enzymes can be present in those fluids. For example, heparanase activity is present in both urine and in plasma. Also, high heparanase activity in blood and plasma is associated with multiple myeloma and may have a role in angiogenesis. A subpopulation of myeloma cells in bone marrow express and secrete heparanase into the bloodstream. Similarly, hyaluronidase is secreted from tumor cells into the bloodstream and into urine. Thus, such fluids may be important in diagnosing or following the progression of cancer. For example, blood, serum, plasma, and/or lymph from a subject with multiple myeloma may be tested for heparanase-degrading activity using an inventive method.

Glycosaminoglycan-degrading enzymes such as hyaluronidase may also be present in blood, plasma, serum, lymph, and/or urine as the result of an infection with a pathogenic species (such as bacteria) that produces glycosaminoglycan-degrading enzymes. Therefore, the provided assays may be useful in diagnosing or following the progression of an infection. Similarly, glycosaminoglycan-degrading enzymes may be present in such fluids as a result of a bite from an organism whose venom contains a glycosaminoglycan-degrading enzyme. Also, glycosaminoglycans including chondroitin sulfate and dermatan sulfate are also known to be secreted in urine.

In certain embodiments of the invention, the biological sample is ascites fluid. As discussed above, glycosaminoglycan-degrading enzymes are secreted from tumor cells, and ascites fluid often contains proteins and other products secreted from tumor cells.

In certain embodiments of the invention, the biological sample is saliva. For example, hyaluronidases produced by oral pathogens may play a role in periodontal disease. Thus, it may be desirable to assay glycosaminoglycan-degrading activity such as that of hyaluronidase in saliva.

In certain embodiments of the invention, the biological sample is synovial fluid, which is known to contain glycosaminoglycans such as chondroitin sulfate, keratan sulfate, and hyaluronan. Levels of such glycosaminoglycans in synovial fluids may be altered in [symbol] glycosaminoglycan-degrading activity such as that of chondroitinase, keratanase, and/or hyaluronidase in synovial fluid. In certain embodiments of the invention, all three activities (that of chondroitinase, keratanase, and hyaluronidase) are assayed for. In certain embodiments of the invention, only two activities (for example, that of chondroitinase and keratanase, that of chondroitinase and hyaluronidase, or that of keratanase and hyaluronidase) are assayed for. In some embodiments of the invention, only one activity is assayed for.

In certain embodiments of the invention, the biological sample is cerebrospinal fluid. Chondroitinases are known to play a role in peripheral nerve repair as well as in functional recovery of a damaged central nervous system. Hyaluronan, which can also be degraded by some chondroitinases, is found to be elevated in some spinal disorders. Thus, it may be desirable to assay activity of glycosaminoglycan-degrading enzymes such as chondroitinase and/or hyaluronidase in cerebrospinal fluid. In certain embodiments of the invention, both chondroitinase and hyaluronidase activities are assayed for. In certain embodiments of the invention, only one activity (for example, either chondroitinase activity or hyaluronidase activity) is assayed for.

In certain embodiments of the invention, the biological sample is tear fluid, the fluid excreted from tear ducts of the eye. For example, chondroitin sulfate found in tear fluids may participate in wound healing in the eye, and hyaluronan is also found in tear fluids. It may be desirable to assay glycosaminoglycan-degrading activity such as that of chondroitinases and hyaluronidases in tear fluid. In certain embodiments of the invention, both chondroitinase and hyaluronidase activities are assayed for. In certain embodiments of the invention, only one activity (for example, either chondroitinase activity or hyaluronidase activity) is assayed for.

In certain embodiments of the invention, the biological sample is vitreous humor (also known as vitreous fluid, the vitreous body, and the vitreous), the fluid contained in the space between the lens and the retina of the eyeball humans and other vertebrates. Hyaluronan is a major component of vitreous humor and is important in retinal function. Intravitreal injection of hyaluronidase is a potential treatment for vision problems such as those due to vitreous hemorrhage. Thus, it may be desirable to assay the activity of glycosaminoglycan-degrading enzymes such as hyaluronidase in vitreous humor. Alternatively or additionally, hyaluronidase may be injected into the eye (e.g., for therapeutic purposes) and may be assayed before use.

In certain embodiments of the invention, the biological sample is venom, such as that from snakes, fish, bees, wasps, scorpions and spiders. For example, hyaluronidases present in the venom of such organisms may be allergenic in susceptible individuals, and it may be desirable to test for the presence of and/or measure the amount of hyaluronidase activity in venom using hyaluronan-coated particles. Also, hyaluronidases present in venoms may subsequently be present in the bodily fluids from an individual bitten by the venomous organism.

In certain embodiments of the invention, the biological sample is seminal fluid. Proteins that bind glycosaminoglycans such as heparin have been detected in seminal fluid of mammalian males, and such proteins may have a role in fertility. Thus, it may be desirable to assay glycosaminoglycan-degrading activity in seminal fluid.

In certain embodiments of the invention, the biological sample is medium from cell or tissue cultures. In some embodiments of the invention, the biological sample is a cell supernatant or cell lysate derived from cells, such as cells taken as part of a sample from an organism and/or cells grown in culture. Cells derived from a biological sample such as a tissue biopsy or cells grown from established cell lines and/or freshly prepared from tissues or embryos (such as cancer cells, embryonic stem cells, adult stem cells, cancer stem cells, fibroblasts, keratinocytes, etc) can be grown in culture and the medium collected for analysis using the provided methods. A supernatant can also be prepared from such cells using methods known in the art and used with inventive methods for assaying the activity of glycosaminoglycan-degrading enzymes.

Similarly, medium, supernatant, or lysate can also be collected or prepared from cells grown in culture. For example, it may be desirable to assay the activity of a glycosaminoglycan-degrading enzyme in a cell expression system (such as a mammalian, insect, or bacterial, etc cell expression systems). Medium, supernatant, or lysate can also be collected or prepared from cells that are genetically modifiable and/or used in screens of chemicals including small molecules and other potential drugs.

For example, glycosaminoglycan-degrading activity such as that of heparanase can be tested in the cell-culture medium collected from cancer cells, or other cells thought to be malignant, grown in vitro.

### Detection of Glycosaminoglycan-Degrading Enzyme Activity

In some embodiments of the invention, degradation of glycosaminoglycans on the [symbol] aggregation can serve as a measure of glycosaminoglycan-degrading activity. In some embodiments of the invention, aggregation of particles results in a change in the light absorbance of the sample that contains glycosaminoglycan-coated particles. In certain embodiments, the change in light absorbance is a change in the visible spectrum. In certain embodiments of the invention, the change in the visible spectrum results in a color change that is detectable to the naked eye. In certain embodiments, the color change is detectable using standard spectroscopy methods that detect a shift in the visible spectrum of the sample being assayed. This shift may be, for example, detectable directly in the biological sample in which glycosaminoglycan-degrading enzymes are present, and in which the glycosaminoglycan-coated particles are allowed to come into contact with such enzymes. In certain embodiments of the invention, the assay is quantitative.

For example, a red shift in the absorbance maximum of the visible spectrum may be detectable in a biological sample such as serum or plasma to which metallic particles coated with glycosaminoglycans have been contacted. The magnitude of the red shift varies in different embodiments. In certain embodiments of the invention, the absorbance maximum of the visible spectrum shifts from about 542 nm to about 567 nm. In certain embodiments of the invention, the biological sample is serum and the absorbance maximum of the visible spectrum shifts from about 542 nm to about 558 nm. In certain embodiments of the invention, the biological sample is plasma and the absorbance maximum of the visible spectrum shifts from about 542 nm to 561 nm.

In certain embodiments of the invention, particle aggregation leads to changes in light-absorptive and scattering properties that are detectable by, for example, transmission electron microscopy. In certain embodiments of the invention, particle aggregation leads to changes in the intensity of the light signal. The light whose signal intensity is changed may fall in the visible range of the spectrum.

Diseases Associated with Glycosaminoglycan-Degrading Enzymes

Provided methods of assaying the activity of glycosaminoglycan-degrading enzymes can be useful in the diagnosis or other assessment of disease, in particular, human or veterinary disease. For example, some enzymes that degrade glycosaminoglycans have altered expression levels (e.g., decreased or increased with respect to normal physiological levels) in certain disease states. For example, altered hyaluronidase activity is associated with cancer cells and has been found in individuals with prostate cancer, bladder cancer, and [symbol] glycosaminoglycans capable of being degraded by hyaluronidases are used to assay a biological sample from, or a biological sample derived from tissues from, an individual. The individual could be healthy, suspected of having cancer, identified as having an increased risk for cancer, treated for cancer, in remission for cancer, being monitored for recurrence of cancer, etc. The individual could also already be diagnosed as having cancer, and the provided methods may be useful in determining the stage of disease progression by using glycosaminoglycan-degrading activity as an indicator.

Elevated heparanase activity has also been associated with cancers including multiple myeloma, colon cancer, breast cancer, lung cancer, prostate cancer, ovary cancer, and pancreatic tumors. In some embodiments of the invention, particles coated with glycosaminoglycans capable of being degraded by heparanases are used to assay a biological sample from, or a biological sample derived from, tissues or cells from an individual. As described above, the cancer and/or health status of the individual from which the biological sample is extracted or derived from can vary.

Glycosaminoglycan-degrading enzymes such as hyaluronidases are also involved in the spreading of bacterial infections, toxins, and venoms. Such infections may also lead to inflammatory conditions. Thus, the inventive methods could also be used in the diagnosis or other assessment of infectious diseases and inflammatory diseases.

Similarly, aberrant levels of glycosaminoglycans may contribute to and/or indicate autoimmune diseases such as rheumatoid arthritis. Thus, provided methods could also be used in the diagnosis or other assessment of autoimmune diseases.

### Kits

Also provided are kits that may be useful in the diagnosis or monitoring of diseases or infections in which glycosaminoglycan-degrading activity is altered or otherwise implicated, as discussed above. Provided kits generally comprise metallic particles coated with glycosaminoglycans, one or more control samples, and instructions. In certain embodiments of the invention, at least one of the provided control samples contains glycosaminoglycan-degrading activity. In certain embodiments of the invention, a negative control sample (that is, a sample lacking detectable glycosaminoglycan-degrading activity) is provided. In certain embodiments of the invention, the kits comprise several control samples, each containing some amount of glycosaminoglycan-degrading activity and/or no glycosaminoglycan-degrading activity. In certain embodiments of the invention, one or more control samples each comprising a defined quantity of purified enzyme is provided. In [symbol] certain embodiments of the invention, defined quantities of more than one enzyme are included in control samples in the inventive kits. Such control samples may be used as standards for calibration and/or quantitation of samples to be tested. Thus, in certain embodiments, the provided kits allow quantitative assays of glycosaminoglycan-degrading activity.

In certain embodiments of the invention, more than one type of glycosaminoglycan-coated metallic particle is provide in a single kit. For example, some kits comprise more than one type of metallic particle (such as, for example, gold and cadmium sulfide). In certain embodiments of the invention, the kits comprise metallic particles coated with one glycosaminoglycan in addition to other metallic particles coated with another glycosaminoglycan. In certain embodiments of the invention, the kits comprise three, four, five, six, seven, eight, or more than eight different kinds of glycosaminoglycan-coated metallic particles. In certain embodiments, the kits comprise particles each coated with more than one, (e.g., two, three, four, five, six, seven, eight, or more than eight) kind of glycosaminoglycan.

### EXAMPLES

The following examples describe some of the preferred modes of making and practicing the present invention. However, it should be understood that these examples are for illustrative purposes only and are not meant to limit the scope of the invention.

### Example 1

### Glycosaminoglycan-Assisted Synthesis of Soluble Gold Nanoparticles

The experiments in this example show that heparan sulfate assists the synthesis of soluble gold nanoparticles when it is present during the mixing of gold nanoparticle precursors.

Specific conditions that allow efficient generation of soluble, biologically active gold nanoparticles (GNPs) coated with heparan sulfate were discovered after exploring several conditions. 5.8 mg (0.013 mmol) of HAuCl₄.3H₂O (the gold precursor) was first mixed with 4 mg of heparan sulfate I in 50 mL deionized water. The mixture was heated for 60° C. for 15 minutes. 1 mL of aqueous trisodium citrate solution (Na₃C₆H₅O₇.H₂O, 1% w/v) was added to the solution, which was then kept at 60° C. for 60 minutes with continued stirring. The solution was then allowed to cool to room temperature. The solution was then centrifuged to remove unbound polysaccharide from the gold nanoparticles, which were coated with heparan sulfate molecules as a result of previous manipulations. The product of such manipulations, a clear purple solution, is depicted in FIG. 2C.

Similar synthesis experiments were conducted using different conditions. FIG. 2A depicts the result of a reaction between HAuCl₄.3H₂O and trisodium citrate precursors in the absence of polysaccharide, and FIG. 2B depicts the result of a reaction in which HAuCl₄.3H₂O and trisodium citrate precursors were mixed together prior to the addition of polysaccharide. The colorlessness of the solutions depicted in FIGS. 2A and 2B indicate lack of product, whereas the color in the tube depicted in FIG. 2C indicates the presence of a soluble gold nanoparticle product.

### Example 2

### Treatment with Heparinase III Results in Detectable Changes to and Aggregation of Heparan Sulfate-Coated Gold Nanoparticles

The following experiments were conducted to evaluate the potential of using glycosaminoglycan-coated nanoparticles in assays for activity of glycosaminoglycan-degrading enzyme.

Heparan sulfate-coated gold nanoparticles (heparan sulfate-GNPs) synthesized by the scheme described in Example 1 were tested as substrates for glycosaminoglycan-degrading enzyme. Heparan sulfate-GNPs were exposed to bacterial Heparinase III as follows: 2 μL of enzyme (0.7 μg/μL Heparinase III) was added to a cuvette containing a 1.5 mL solution of heparan sulfate-GNPs in phosphate-buffered saline solution (without calcium chloride or magnesium chloride), pH 7.4 at 35° C.

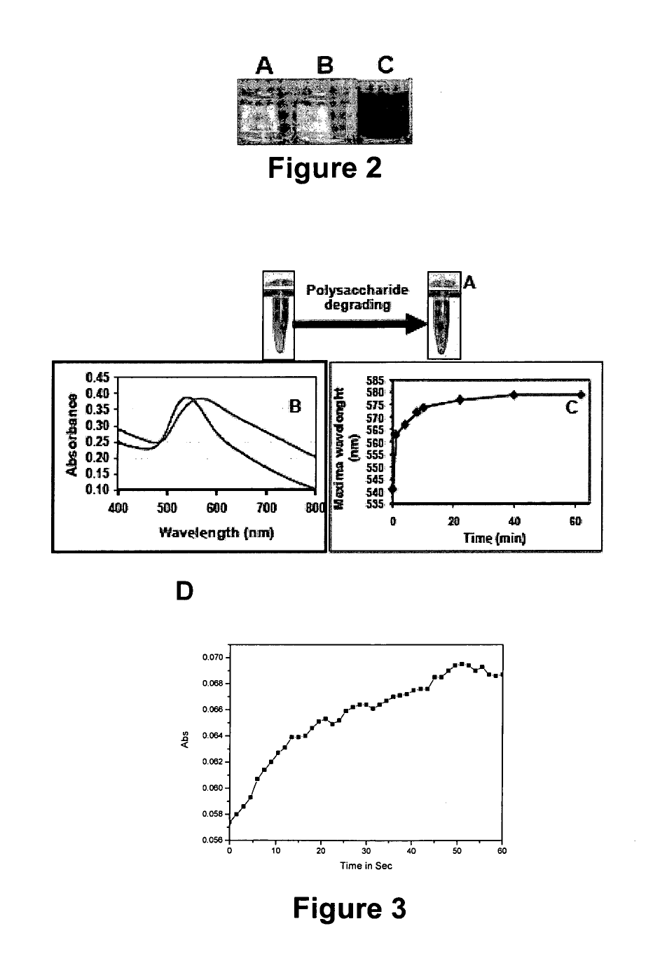

Treatment of the heparan sulfate-GNPs with enzyme resulted in a color change of the solution that is visible to the naked eye (see FIG. 3A). Shown in FIG. 3B are absorbance spectra of the solution before (purple) and after (blue) treatment with enzyme. Treatment with enzyme resulted in a red shift in the visible spectrum from 542 nm to 567 nm. FIG. 3C depicts the change, in absorbance maximum as a function of enzyme treatment time, and FIG. 3D depicts the change in absorbance intensity at the wavelength maximum after addition of 1 μg heparinase.

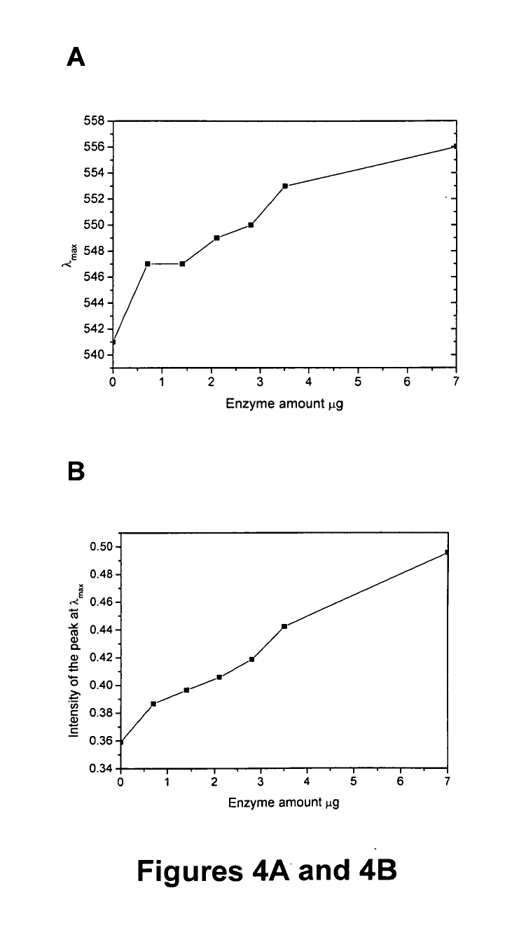

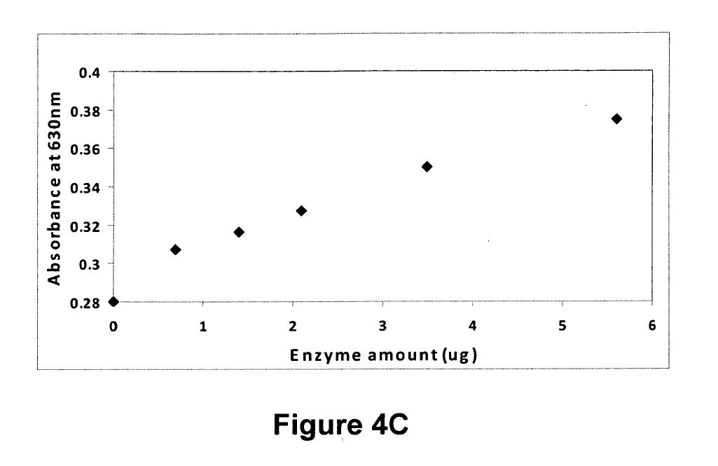

In a separate experiment, varying amounts of enzyme were incubated with heparan sulfate-GNPs. Increasing amounts of enzyme caused increased red shifting of the absorbance maximum peak (see FIG. 4A). FIGS. 4B and 4C show plots of absorbance at λₘₐₓ and 630 nm respectively as a function of enzyme amount.

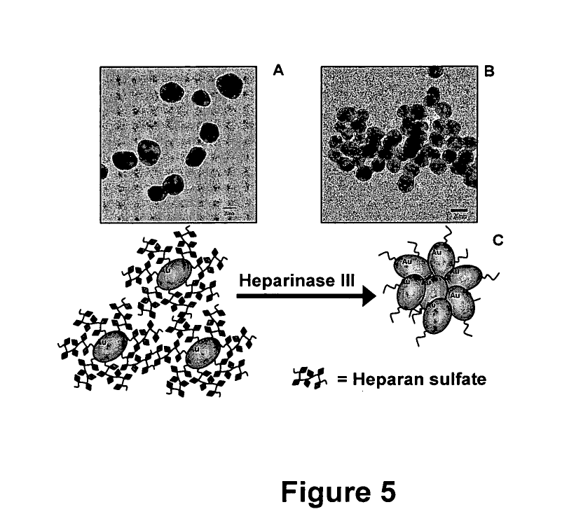

The light-absorptive and scattering properties of GNPs depend on the particle's [symbol] of heparan sulfate-GNPs upon treatment with enzyme was evaluated using Transmission Electron Microscopy (TEM). Shown in FIG. 5 are TEM micrographs of heparan sulfate-GNPs. The average diameter of the metallic portion of heparan sulfate-GNPs was determined by TEM to be about 20 nm±5 nm.

Heparan sulfate-GNPs are well dispersed before treatment with enzyme (FIG. 5A), but cluster together into aggregates after enzyme treatment (FIG. 5B). A schematic representation of aggregation of GNPs upon enzyme treatment is depicted in FIG. 5C.

These experiments demonstrate that heparan sulfate chains of heparan sulfate-GNPs synthesized by the provided methods can be degraded by Heparinase III in solution. Such degradation leads to visibly detectable changes and also leads to aggregation of the GNPs. In addition to being suitable for use in an enzyme activity assay, heparan sulfate-GNPs were stable. They can be incubated in water at 4° C. for at least two weeks without apparent degradation.

### Example 3

### Treatment of Heparan Sulfate-GNPs with Heparinase III in the Presence of Serum and Plasma Results in Detectable Changes to the Nanoparticles

The following experiments were conducted to evaluate the viability of the presently disclosed methods of assaying activity of glycosaminoglycan-degrading enzyme in the presence of biological fluids.

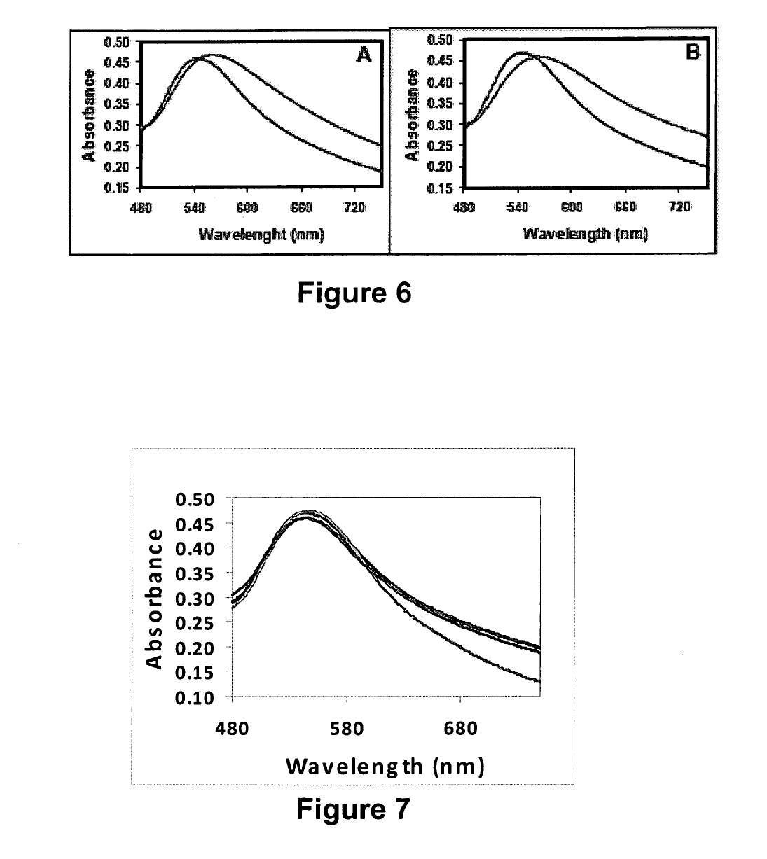

Assays for Heparinase III activity were conducted in serum and plasma samples using heparan sulfate-GNPs. Each sample contained 100 μL of serum or plasma spiked with 2 μl of enzyme (0.7 μg/μL Heparinase III) and 0.5 mL heparan sulfate-GNP solution (generated with 0.08 μg/μL heparan sulfate and 0.13 μmol Au). As in Example 2, the absorbance spectra were recorded. The results are depicted in FIG. 6. A red shift from 542 nm to 588 nm was observed upon enzyme treatment of heparan sulfate-GNPs in plasma (FIG. 6A). A red shift from 542 nm to 561 nm was observed upon enzyme treatment of heparan sulfate-GNPs in serum (FIG. 6B).

To determine the potential robustness of the assay in different fluids, the photophysical properties of heparan sulfate gold nanoparticles were examined in a variety of fluids. For each sample, 1 mL of serum (purple), 1 mL, 1 mL of plasma (red) or 1 mL of 10% w/v BSA (blue) or 1 mL of PBS buffer pH 7.4 (green) were added to a solution containing 1.5 mL of 51.2 μg/mL heparan sulfate-GNP in water and 0.4 mL of PBS buffer pH 7.4. As shown in FIG. 7, heparan sulfate-GNP showed a roughly consistent absorbance spectrum in serum, plasma, 10% BSA, PBS, and water.

These experiments demonstrate that treatment of heparan-sulfate-GNPs with Heparinase III in serum and plasma results in detectable changes in absorbance in the visible spectra of such samples, and that heparan-sulfate GNPs may be useful in assays in other fluids as well.

### Example 4

### Polysaccharide-Assisted Synthesis of Soluble Cadmium Sulfide (CdS) Nanoparticles

The following experiments were conducted to illustrate a method of synthesizing soluble CdS nanoparticles coated with glycosaminoglycans. All chemical reagents used in syntheses were analytical grade, purchased from Sigma-Aldrich, and used without further purification. Cadmium and sulfur precursors were mixed together in 1:1 molar ratios at room temperature with vortex agitation.

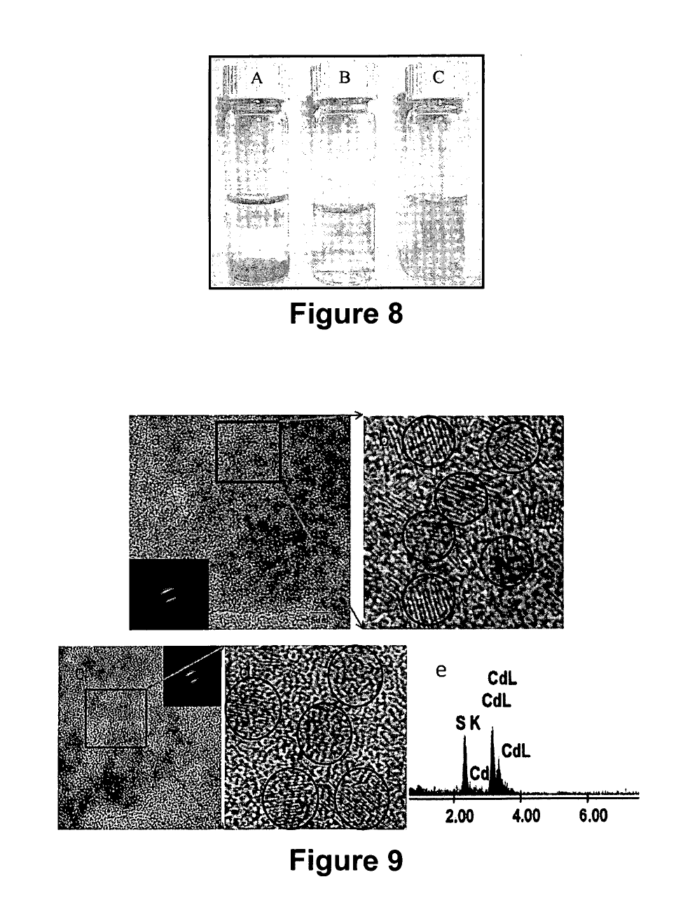

In a typical synthesis, 6 mg of Cd(NO₃)₂ was first mixed with polysaccharide in 2 mL deionized water. The polysaccharide used in each reaction was either chitosan oligosaccharide lactate (15 mg) or Heparan sulfate I (4 mg). After mixing the cadmium precursor with polysaccharide, 2 mL (6 mg) of aqueous NaS₂ solution was added to the solution and the solution was agitated by vortexing. The solution immediately turned yellow, indicating that soluble CdS nanoparticles had formed. Shown in FIG. 8 are photographs of CdS nanoparticles in water after reactions (A) without any kind of polysaccharide, (B) with assistance of heparan sulfate I, and (C) with assistance of chitosan oligosaccharide. The clear yellow color of the tubes depicted in (B) and (C) indicate the presence of water-soluble CdS nanoparticles. The reaction without polysaccharide, depicted in (A), formed a precipitate with yellow floccules that settled onto the bottom of the tubes, indicating that the product in that reaction was not water-soluble.

To investigate the mechanism for the role of polysaccharides in assisting the synthesis of CdS nanoparticles, additional synthesis reactions were carried out in which cadmium and sulfate precursors were first mixed together. Polysaccharides were subsequently added to the solution and mixed. Such a reaction scheme did not yield soluble CdS nanoparticle product, as the solution contained yellow flocculate precipitates that did not go into solution upon the addition of polysaccharide. This suggests that, in some conditions, polysaccharides need to be mixed with Cd precursors prior to addition of the sulfate precursors in order to generate a soluble product. The polysaccharides may assist in the synthesis of soluble product by binding to Cd²⁺ via non-covalent bonds and preventing the formation of bulk semiconductor.

These experiments demonstrate that the polysaccharides heparan sulfate and chitosan oligosaccharide lactate assist the synthesis of soluble CdS nanoparticles when they are mixed with Cd precursors before the addition of sulfur precursors. In addition, CdS nanoparticles coated with glycosaminoglycans or other polysaccharide chains can be synthesized using the procedure described in this Example.

### Example 5

### Physical Properties of CdS Nanoparticles Synthesized with the Assistance of Chitosan Oligosaccharide Lactate or Heparan Sulfate

The purpose of these experiments is to examine the physical properties of CdS nanoparticles synthesized as described in Example 5.

To determine their morphology, dimension and size distribution, CdS nanoparticles synthesized with the assistance of chitosan oligosaccharide lactate or with heparan sulfate were imaged by TEM by a JEOL model 2011 instrument operated at 200 kV. Samples for TEM analysis and energy dispersive analysis of X-ray (EDAX) were prepared by placing a drop of the sample in water on Formvar-carbon TEM grids. Excess solvent was allowed to dry completely at room temperature.

FIGS. 9A and 9C show TEM images of CdS nanoparticles as prepared with the assistance of chitosan oligosaccharide lactate and heparan sulfate I respectively, with higher magnifications shown in FIGS. 9B and 9D. A typical EDAX spectrum of synthesized CdS nanoparticles is depicted in FIG. 9E. The average size of the nanoparticles was measured from TEM images and were found to be 4 nm±0.3 nm. As shown in FIGS. 9B and 9D, the CdS nanoparticles are highly crystallized.

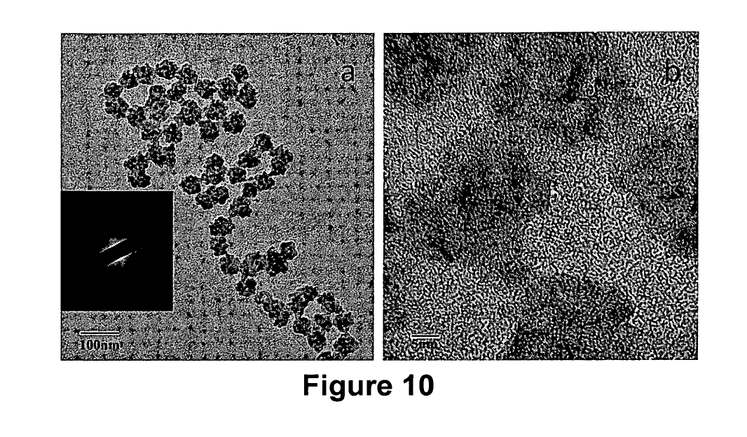

To examine the morphology of CdS nanoparticles at higher temperatures, heparan sulfate-coated CdS nanoparticles were heated at an annealing temperature of 60° C. for 60 minutes. As shown in the TEM images in FIGS. 10A and 10B, smaller nanocrystals aggregated together, with an average aggregate diameter of 50 nm.

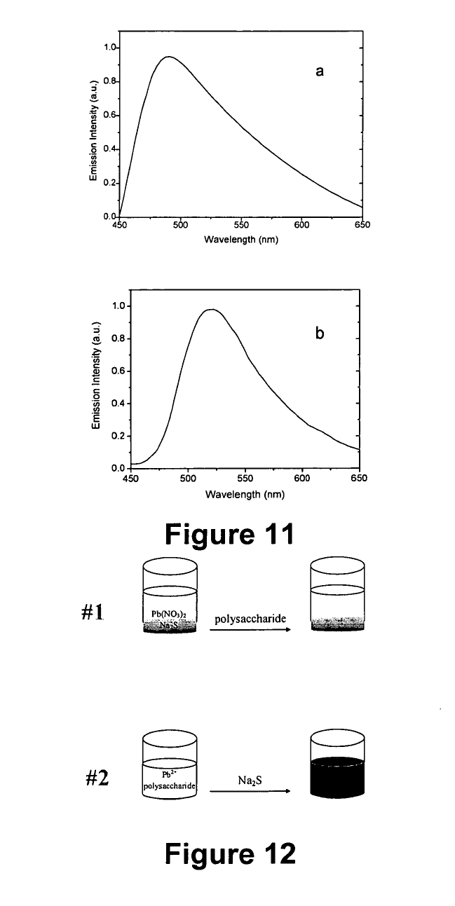

Optical properties of CdS nanoparticles were also examined. The fluorescence spectra were recorded on a Jobin Yvon FluoroMax-2 photoluminescence spectrometer. Spectra were obtained with the slits set at 2 nm and an integration time of one second. CdS nanoparticles were analyzed in water with samples placed in quartz cuvettes (Starna Cells). Shown in FIG. 11 are the fluorescence spectra of CdS nanoparticles synthesized in the presence of heparan sulfate, as synthesized (A) and after annealing at 60° C. for 60 minutes (B). A shift in peak position was observed: the emission peak was 492 nm (2.73 eV) for freshly synthesized CdS nanoparticles and 520 nm (2.50 eV) for CdS nanoparticles after annealing.

### Example 6

### Polysaccharide-Assisted Synthesis of Soluble Lead Sulfide (PbS) Nanoparticles

The following experiments were conducted to illustrate a method of synthesizing soluble lead sulfide (PbS) nanoparticles coated with glycosaminoglycans. Two different reaction schemes (represented by the diagrams in FIG. 12) were tested for synthesizing PbS nanoparticles. In process #1, Pb(NO₃)₂ and Na₂S were mixed together first, and then polysaccharide was added to the solution. In process #2, Pb(NO₃)₂ was mixed with polysaccharide first, then Na₂S was added to the solution.

All polysaccharides used in syntheses were purchased from Sigma-Aldrich, and used without further purification. 12 mM aqueous stock solutions of polysaccharide were freshly prepared with deionized water. The lead and sulfur precursors, Pb(NO₃)₂ (99%) and Na₂S (98%) were also purchased from Sigma-Aldrich. Aqueous stock solutions of 10 mM Pb(NO₃)₂ and 10 mM Na₂S.9H₂O were freshly prepared.

Lead and sulfur precursors were mixed together in 1:1 molar ratios in 2 mL eppendorf tubes at room temperature with vortex agitation. In a typical synthesis reaction using process #1, 100 μL of 10 mM Pb(NO₃)₂ was mixed with 100 μL of 10 mM Na₂S.9H₂O first, forming a black precipitate, indicating the formation of PbS. Then 250 mL of 12 mM polysaccharide was added to the reaction, and the solution was agitated by vortexing.

In a typical synthesis reaction using process #2, 100 μL of 10 mM Pb(NO₃)₂ was added to 250 μL of 12 mM polysaccharide and mixed, followed by a 5 minute incubation. Then 100 μL of 10 mM Na₂S.9H₂O was added to the reaction tube. The solution immediately turned from transparent to red-brown, indicating the formation of PbS nanoparticles.

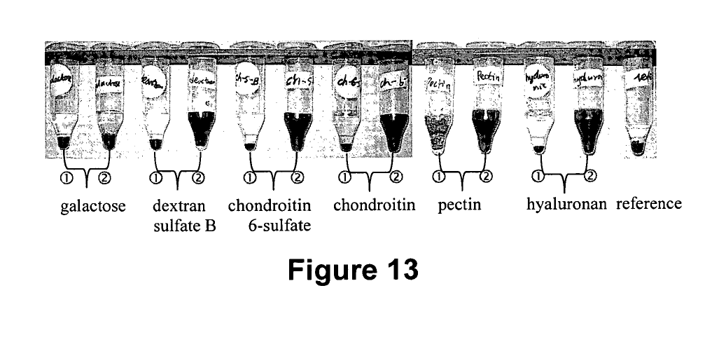

Syntheses were carried out by process #1 and process #2 using galactose, dextran sulfate B, chondroitin 6-sulfate (chondroitin sulfate A), chondroitin acid, pectin, and hyaluronan. A reference reaction was also carried out using no polysaccharide or glycosaminoglycan. As shown in FIG. 13, products from reactions using process #1 were less soluble (i.e., more PbS existed in a precipitate) than products from reactions using process #2. Binding of polysaccharide or glycosaminoglycans to lead precursors prevented formation of bulk semiconductor. The results shown in FIG. 13 also indicate that long [symbol] chondroitin acid, and hyaluronan assist in the synthesis of a PbS product that is more soluble than short chain polysaccharides such as galactose do. Under the reaction conditions described in this Example, the glycosaminoglycans and other long chain polysaccharides serve as a competent ligand for nucleation, growth, and capping of soluble PbS nanoparticles. Results of synthesis reactions are summarized in Table 1 below:

TABLE 1
Different physical properties for the synthesized
PbS w/different kinds of polysaccharides.

PbS

| | color | solubility |
|---|---|---|
| Without polysaccharide | Black | Black precipitation |
| Pectin | Dark brown | Clear solution |
| Heparin | Dark brown | Clear solution |
| hyaluronic acid | Dark brown | Clear solution |
| Chondroitin 6-sulfate | Dark brown | Clear solution |
| dextratan sulfate | Dark brown | Clear solution |
| Dextran | Dark brown | Clear solution |
| Chondroitin sulfate B | Dark brown | Clear solution |

These experiments demonstrate that various polysaccharides assist the synthesis of soluble PbS nanoparticles when they are mixed with lead precursors before addition of sulfur precursors.

### Example 7

### Physical Properties of PbS Nanoparticles Synthesized with the Assistance of Polysaccharides or Glycosaminoglycans

The purpose of these experiments is to examine the physical properties of PbS nanoparticles synthesized as described in Example 6.

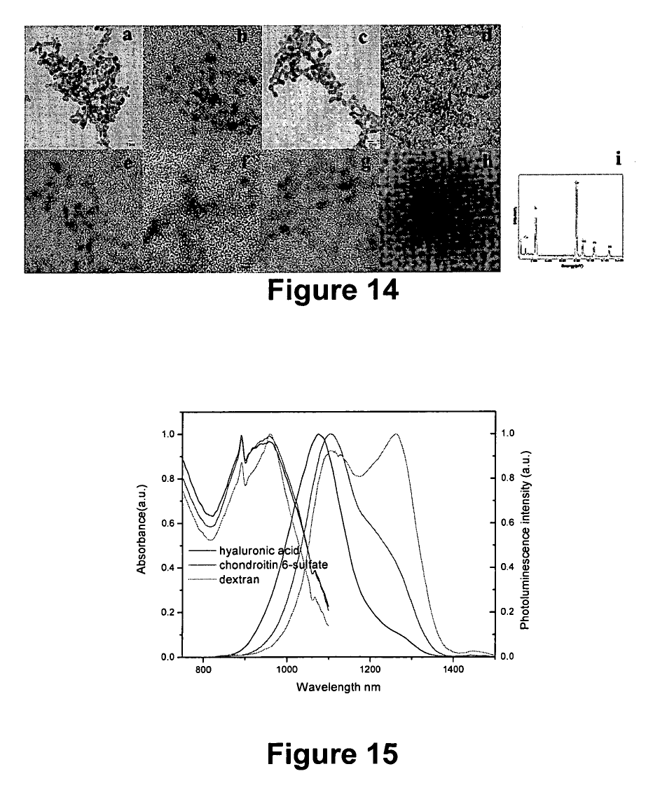

Products synthesized by process #2 in Example 6 were imaged by TEM for their morphology and found to be approximately 5 nm spherical nanoparticles. As shown in FIG. 14A, when PbS nanoparticles were synthesized without any polysaccharide whatsoever, they formed aggregates. Synthesis of PbS nanoparticles in the presence of galactose led to PbS nanoparticles (shown in FIG. 14B) that were slightly more soluble and less aggregated compared to the PbS nanoparticles synthesized without any polysaccharide.

PbS nanoparticles synthesized in the presence of longer chain polysaccharides or glycosaminoglycans (shown in FIGS. 14C-G) were even better separated from each other than the PbS nanoparticles synthesized in the presence of galactose. The EDAX spectrum of a typically synthesized PbS nanoparticle, shown in FIG. 14I, confirmed that the nanoparticles were indeed composed of lead and sulfide.

To confirm that the PbS nanoparticles were successfully coated on the surface with glycosaminoglycans or other polysaccharide chains, the zeta potential (i.e., net particle charge) of PbS nanoparticles synthesized with various glycosaminoglycans or polysaccharide chains was measured. The zeta potential is expected to be different than the zeta potential of uncoated PbS nanoparticles due to the charge of the glycosaminoglycans or polysaccharides coating the surface. For example, chondroitin sulfate C, dermatan sulfate, heparin, and hyaluronan are highly negatively charged. As shown in Table 2, the zeta potential for PbS nanoparticles synthesized with dextran, chondroitin sulfate A, chondroitin sulfate B (dermatan sulfate), heparin, pectin, and hyaluronan differed greatly from the zeta potential for the reference sample (PbS nanoparticles synthesized without any polysaccharides.) For example, the zeta potential of reference PbS nanoparticles was −3.73, whereas the zeta potential of PbS nanoparticles synthesized with chondroitin sulfate C and dermatan sulfate was −62.75 and −65.71, respectively. Thus, PbS nanoparticles synthesized by process #2 with glycosaminoglycans and other polysaccharides were coated on their surfaces with such glycosaminoglycans and other polysaccharides.

TABLE 2
Zeta potential for different polysaccharides coated PbS samples
compared with reference sample (no polysaccharide). A negative zeta
potential means that the particle has a net negative surface charge,
and vice versa.

| | | | | | | | Hyaluronic |
|---|---|---|---|---|---|---|---|
| | Ref | Dextran | Chondroitin C | Chondroitin B | Heparin | Pectin | acid |
| Zeta | −3.73 | 0.19 | −62.75 | −65.51 | −48.4 | −28.8 | −53.05 |
| potential | | | | | | | |
| (mv) | | | | | | | |

The optical properties of PbS nanoparticles coated with hyaluronan, chondroitin sulfate A, and dextran were explored by UV-Vis and fluorescence spectroscopy using techniques as described in Example 5. As shown in FIG. 15, PbS nanoparticles exhibit well-defined excitation peaks in their absorption spectra and band-edge luminescence that can be tuned throughout the near-infrared spectrum. As for the fluorescent spectra (shown on the right side of FIG. 15), the peaks show red-shifting for PbS nanoparticles synthesized [symbol] dextran. Without wishing to be bound by any theory, this red-shifting is likely associated with trap-state emission, which accounts for the weakened, red-shifted luminescence. The second feature in the spectra (the shoulder) is likely due to the size distribution or linkage of particles by polysaccharide.

These experiments explored the properties of synthesized PbS nanoparticles and show that PbS nanoparticles synthesized using provided methods are soluble and coated with glycosaminoglycans and other polysaccharide chains.

### EQUIVALENTS AND SCOPE

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A metallic particle coated with a glycosaminoglycan, wherein the largest dimension of the metallic portion of the particle is smaller than 1 μm.

2. The particle of claim 1, wherein the particle comprises gold.

3. The particle of claim 1, wherein the particle comprises cadmium.

4. The particle of claim 1, wherein the particle comprises lead.

5. The particle of claim 1, wherein the particle is a gold particle.

6. The particle of claim 1, wherein the particle is a CdS particle.

7. The particle of claim 1, wherein the particle is a PbS particle.

8. The particle of claim 1, wherein the glycosaminoglycan is degradable by an enzyme whose expression level is altered in a disease state.

9. The particle of claim 8, wherein the glycosaminoglycan is degradable by an enzyme whose expression level is altered in an infectious disease.

10. The particle of claim 8, wherein the glycosaminoglycan is degradable by an enzyme whose expression level is altered in an inflammatory disease.

11. The particle of claim 8, wherein the glycosaminoglycan is degradable by an enzyme whose expression level is altered in cancer.

12. The particle of claim 1, wherein the glycosaminoglycan is degradable by an enzyme whose activity is altered in a disease state.

13. The particle of claim 12, wherein the glycosaminoglycan is degradable by an enzyme whose activity is altered in infectious disease.

14. The particle of claim 12, wherein the glycosaminoglycan is degradable by an enzyme whose activity is altered in inflammatory disease.

15. The particle of claim 12, wherein the glycosaminoglycan is degradable by an enzyme whose activity is altered in cancer.

16. The particle of claim 1, wherein the glycosaminoglycan is a glycosaminoglycan degradable by an enzyme secreted from a cancer cell.

17. The particle of claim 1, wherein the glycosaminoglycan is selected from the group consisting of chondroitin, chondroitin sulfate, dermatan, dermatan sulfate, keratan, keratan sulfate, heparin, heparan sulfate, and hyaluronan.

18. The particle of claim 2, wherein the glycosaminoglycan is heparan sulfate.

19. The particle of claim 2, wherein the glycosaminoglycan is hyaluronan.

20. The particle of claim 1, wherein the particle is roughly spherical in shape.

21. The particle of claim 20, wherein the diameter of the metallic portion of the particle ranges from about 1 nm to about 500 nm.

22. The particle of claim 21, wherein the diameter of the metallic portion of the particle ranges from about 3 nm to about 50 nm.

23. A method of assaying glycosaminoglycan-degrading activity, the method comprising steps of:
(a) providing metallic particles coated with a glycosaminoglycan, wherein the glycosaminoglycan is capable of being degraded by the activity being assayed for;
(b) contacting the particles with a sample under suitable conditions to allow the glycosaminoglycan-degrading activity in the sample to degrade the glycosaminoglycans on the surface of the particles; and
(c) determining the glycosaminoglycan-degrading activity in the sample by evaluating the aggregation of the particles.

24. The method of claim 23, wherein the glycosaminoglycan-degrading activity is due to an enzyme.

25. The method of claim 23, wherein the metallic particles comprise gold.

26. The method of claim 23, wherein the metallic particles are gold particles.

27. The method of claim 24, wherein the glycosaminoglycan-degrading enzyme is a polysaccharide lyase.

28. The method of claim 24, wherein the glycosaminoglycan-degrading enzyme is a glycoside hydrolase.

29. The method of claim 24, wherein the glycosaminoglycan-degrading enzyme is selected from the group consisting of heparanases, heparinases, hyaluronidases, chondroitinases, dermatanases, and keratanases.

30. The method of claim 24, wherein the glycosaminoglycan-degrading enzyme is a heparanase.

31. The method of claim 24, wherein the glycosaminoglycan-degrading enzyme is a hyaluronidase.

32. The method of claim 23, wherein the sample is obtained during production of a therapeutic formulation, commercial product, or both.

33. The method of claim 32, wherein the therapeutic formulation comprises a glycosaminoglycan-degrading enzyme.

34. The method of claim 23, wherein the sample is a biological sample.

35. The method of claim 34, wherein the biological sample is selected from the group consisting of: serum, plasma, blood, lymph, ascites fluid, urine, saliva, synovial fluid, cerebrospinal fluid, tears, vitreous humor, seminal fluid, cell supernatant, cell lysate, and cell culture medium.

36. The method of claim 35, wherein the biological sample is selected from the group consisting of serum, plasma, blood, and urine.

37. The method of claim 23, wherein the step of determining comprises monitoring a change in light absorbance.

38. The method of claim 23, wherein monitoring a change in light absorbance comprises determining a shift in wavelength of the peak of the visible spectrum of the particles.

39. The method of claim 38, wherein the shift in the wavelength of the peak is at least 3 nm.

40. The method of claim 39, wherein the shift is a red shift.

41. The method of claim 23, wherein the step of determining comprises monitoring a change in the light signal intensity.

42. A method of preparing glycosaminoglycan-coated gold particles, the method comprising steps of:
(a) mixing a gold precursor with a glycosaminoglycans in water;
(b) heating the resulting solution;
(c) adding a reducing agent to the gold-glycosaminoglycan solution with stirring; and
(d) optionally, isolating the resulting glycosaminoglycan-coated gold particles.

43. The method of claim 42, wherein the gold precursor is HAuCl₄.

44. The method of claim 42, wherein the reducing agent is selected from the group consisting of citrate solutions, sodium borohydride solutions, thiocyanate solutions, phosphorus solutions, ascorbic acid solutions, and ethyl alcohol solutions.

45. The method of claim 44, wherein the reducing agent is a citrate solution.

46. The method of claim 42, wherein the glycosaminoglycan-coated gold particles are soluble in water.

47. The method of claim 42, wherein steps (a)-(d) are each carried out at temperatures lower than 80° C.

48. A method of preparing glycosaminoglycan-coated cadmium sulfide particles, the method comprising steps of:
(a) mixing a cadmium precursor with glycosaminoglycan in water; and
(b) adding a source of sulfide to the cadmium-glycosaminoglycan solution.

49. The method of claim 48, wherein the glycosaminoglycan-coated cadmium sulfide particles are soluble in water.

50. The method of claim 48, wherein steps (a) and (b) are each carried out at temperatures lower than 80° C.

51. A method of preparing glycosaminoglycan-coated lead sulfide particles, the method comprising steps of:
(a) mixing a lead precursor with glycosaminoglycans in water;
(b) incubating the resulting mixture at room temperature; and
(c) adding a source of sulfide to the lead-glycosaminoglycan solution.

52. The method of claim 51, wherein the glycosaminoglycan-coated lead sulfide particles are soluble in water.

53. The method of claim 51, wherein steps (a)-(c) are each carried out at temperatures lower than 80° C.

54. A kit for assaying glycosaminoglycan-degrading activity, comprising
(a) metallic particles coated with glycosaminoglycans;
(b) a control sample containing glycosaminoglycan-degrading activity; and
(c) instructions for use.

55. The kit of claim 54, further comprising a control sample not containing glycosaminoglycan-degrading activity.

56. A particle coated with a glycosaminoglycan selected from the group consisting of chondroitin, chondroitin sulfate, dermatan, dermatan sulfate, keratan, keratan sulfate, heparin, heparan sulfate, and hyaluronan, wherein the particle comprises gold and wherein the largest dimension of the metallic portion (core) of the particle is less than approximately 1 μm.

57. The particle of claim 56, wherein the particle is coated with heparan sulfate.

58. A particle coated with a glycosaminoglycan, wherein particle comprises gold and is roughly spherical in shape, and wherein the diameter of the metallic portion of the

[symbol]

59. A particle coated with a glycosaminoglycan, wherein particle comprises gold and is roughly spherical in shape, and wherein the diameter of the metallic portion of the particle is greater than 20 nm and smaller than 1 μm.

60. The particle of claim 59, wherein the diameter of the metallic portion of the particle is greater than 21 nm.

61. The particle of claim 60, wherein the diameter of the metallic portion of the particle is greater than 22 nm.

62. The particle of claim 1, wherein the glycosaminoglycan comprises more than 5 disaccharide units in a single molecule.

63. The particle of claim 1, wherein the glycosaminoglycan comprises more than 10 disaccharide units in a single molecule.

64. The particle of claim 1, wherein the glycosaminoglycan comprises more than 15 disaccharide units in a single molecule.

65. The particle of claim 1, wherein the glycosaminoglycan comprises more than 25 disaccharide units in a single molecule.

66. The particle of claim 1, wherein the glycosaminoglycan comprises more than 50 disaccharide units in a single molecule.

67. The particle of claim 1, wherein the glycosaminoglycan comprises more than 100 disaccharide units in a single molecule.

68. The particle of claim 1, wherein the glycosaminoglycan comprises more than 200 disaccharide units in a single molecule.

69. The particle of claim 1, wherein the glycosaminoglycan comprises more than 500 disaccharide units in a single molecule.

70. The particle of claim 1, wherein the glycosaminoglycan comprises more than 1000 disaccharide units in a single molecule.
